(19) United States
(12) Patent Application Publication
(10) Pub. No.: US 2018/0360299 A1
(43) Pub. Date: Dec. 20, 2018

(54) IMAGING APPARATUS, IMAGING METHOD, AND MEDICAL OBSERVATION EQUIPMENT

(71) Applicant: SONY CORPORATION, Tokyo (JP)

(72) Inventors: Koichiro KISHIMA, Kanagawa (JP); Takuya KISHIMOTO, Tokyo (JP); Akio FURUKAWA, Tokyo (JP); Hiroshi MAEDA, Kanagawa (JP)

(73) Assignee: SONY CORPORATION, Tokyo (JP)

(21) Appl. No.: 16/061,575

(22) PCT Filed: Feb. 22, 2017
(86) PCT No.: PCT/JP2017/006666

§ 371 Date: Jun. 12, 2018

(30) Foreign Application Priority Data
Mar. 28, 2016 (JP) 2016-063783
Dec. 22, 2016 (JP) 2016-249396

(51) Int. Cl. A61B 1/07; A61B 1/00; A61B 1/04; A61B 1/06; A61B 5/00

(52) U.S. Cl. CPC A61B 1/07; A61B 2562/0233; A61B 1/042; A61B 1/063; A61B 1/0646; A61B 1/043; A61B 1/0638; A61B 5/0066; A61B 5/0091; A61B 5/0084; A61B 5/0071; A61B 1/00006; A61B 1/0669; A61B 1/00126; A61B 1/00096

(57) ABSTRACT

A medical imaging system (10) including an optical branching device (101) having a plurality of optical paths for guiding light for imaging a target comprising a biotissue, each of the optical paths corresponding to an optical port connectable to an external device for imaging, wherein at least one path of the plurality of optical paths is configured both to guide the irradiation light to the biotissue and to guide light from the biotissue, and wherein the optical branching device includes a plurality of prisms and at least one joint surface.

### CROSS REFERENCE TO RELATED APPLICATIONS

This application claims the benefit of Japanese Priority Patent Application JP 2016-063783 filed Mar. 28, 2016, and Japanese Priority Patent Application JP 2016-249396 filed Dec. 22, 2016, the entire contents of each of which are incorporated herein by reference.

### TECHNICAL FIELD

The present disclosure relates to an imaging apparatus, an imaging method and medical observation equipment.

### BACKGROUND ART

In operations for breast cancer, sentinel lymph node dissection is performed. There are multiple methods of identifying the position of the sentinel lymph node. One of the methods is to identify a lymphatic vessel by administering a liquid having lymph transferability which modifies a radioactive material through the lymphatic vessel to detect gamma rays radiated from the radioactive material (Radio Isotope: RI method). Another method is to identify a lymphatic vessel using a dye having lymph transferability (color dyeing method).

In addition to these methods, a method of identifying a lymphatic vessel by administering indocyanine green (ICG), which is a fluorogenic reagent having lymph transferability, to the body to perform fluorescence observation in wavelength bands that are not visible to the naked eyes has been recently proposed as a new sentinel lymph node identification method. This sentinel lymph node identification method using ICG has recently been used in actual operations for breast cancer (refer to Non-Patent Literature 1 below, for example).

### CITATION LIST

### Non Patent Literature

NPL 1: S. L. Troyan et al., “The FLARE™ Intraoperative Near-Infrared Fluorescence Imaging System: A First-in-Human Clinical Trial in Breast Cancer Sentinel Lymph Node Mapping”, Annals of Surgical Oncology, 2009, Volume 16, issue 10, p. 2943-2952.

### SUMMARY

### Technical Problem

However, the system of the aforementioned Non-Patent Literature 1 used for actual medical practice has a very large imaging apparatus, as illustrated in FIG. 2 of the literature, and thus it is important to promote miniaturization of the imaging apparatus.

In addition, attempts to digitally image biotissue that is an observation object and display the resulting image on a display screen are being made, for example, for medical observation equipment such as an endoscope and an arthroscope as well as the aforementioned medical observation equipment used to identify the sentinel lymph node in operations for breast cancer. With respect to such medical observation equipment, it is also important to miniaturize the medical observation equipment (particularly, part corresponding to an imaging apparatus).

Accordingly, the present disclosure proposes a miniaturized imaging apparatus which is used when an imaging target such as biotissue is imaged, a method of imaging an imaging target using the imaging apparatus, and medical observation equipment in view of the above circumstances.

### Solution to Problem

According to the present embodiments there is described a medical imaging system, including an optical branching device having a plurality of optical paths for guiding light for imaging a target comprising a biotissue, each of the optical paths corresponding to an optical port connectable to an external device for imaging, wherein at least one path of the plurality of optical paths is configured both to guide the irradiation light to the biotissue and to guide light from the biotissue, and wherein the optical branching device includes a plurality of prisms and at least one joint surface.

According to another embodiment there is described an optical branching device, including: a plurality of optical paths for guiding light for imaging a target comprising a biotissue, each of the optical paths corresponding to an optical port connectable to an external device for imaging, wherein at least one path of the plurality of optical paths is configured both to guide the irradiation light to the biotissue and to guide light from the biotissue, and wherein the optical branching device includes a plurality of prisms and at least one joint surface.

### Advantageous Effects of Invention

As described above, according to the present disclosure, a further miniaturized imaging apparatus, a method of imaging an imaging target using the imaging apparatus, and medical observation equipment can be realized.

Note that the effects described above are not necessarily limitative. With or in the place of the above effects, there may be achieved any one of the effects described in this specification or other effects that may be grasped from this specification.

### BRIEF DESCRIPTION OF DRAWINGS

FIG. 1A is an explanatory diagram schematically illustrating an example of a configuration of an imaging apparatus according to an embodiment of the present disclosure.

FIG. 1B is an explanatory diagram schematically illustrating an example of the configuration of the imaging apparatus according to the embodiment.

FIG. 1C is an explanatory diagram schematically illustrating an example of the configuration of the imaging apparatus according to the embodiment.

FIG. 1D is an explanatory diagram schematically illustrating an example of the configuration of the imaging apparatus according to the embodiment.

FIG. 2A is an explanatory diagram schematically illustrating an example of a configuration of an irradiation position control unit included in the imaging apparatus according to the embodiment.

FIG. 2B is an explanatory diagram schematically illustrating an example of the configuration of the irradiation position control unit included in the imaging apparatus according to the embodiment.

FIG. 3 is an explanatory diagram schematically illustrating an example of the configuration of the imaging apparatus according to the embodiment.

FIG. 4 is an explanatory diagram of a branching optical system included in the imaging apparatus according to the embodiment.

FIG. 5 is an explanatory diagram of an example of application to an endoscope/arthroscope of the imaging apparatus according to the embodiment.

FIG. 6 is an explanatory diagram of a table of examples of the configuration of the imaging apparatus according to the embodiment.

FIG. 7 is an explanatory diagram schematically illustrating an example of the configuration of the imaging apparatus according to the embodiment.

FIG. 8 is an explanatory diagram schematically illustrating an example of the configuration of the imaging apparatus according to the embodiment.

FIG. 9 is an explanatory diagram schematically illustrating an example of the configuration of the imaging apparatus according to the embodiment.

FIG. 10 is an explanatory diagram schematically illustrating an example of the configuration of the imaging apparatus according to the embodiment.

FIG. 11 is an explanatory diagram schematically illustrating an example of the configuration of the imaging apparatus according to the embodiment.

FIG. 12 is an explanatory diagram schematically illustrating an example of the configuration of the imaging apparatus according to the embodiment.

FIG. 13 is an explanatory diagram schematically illustrating an example of a visible light imaging device in the imaging apparatus according to the embodiment.

FIG. 14 is an explanatory diagram schematically illustrating another example of the branching optical system in the imaging apparatus according to the embodiment.

FIG. 15 is a block diagram schematically illustrating an example of a configuration of an arithmetic processing apparatus included in the imaging apparatus according to the embodiment.

FIG. 16 is an explanatory diagram schematically illustrating an example of image processing in the arithmetic processing apparatus according to the embodiment.

FIG. 17 is an explanatory diagram schematically illustrating an example of data analysis processing in the arithmetic processing apparatus according to the embodiment.

FIG. 18 is an explanatory diagram schematically illustrating an example of data analysis processing in the arithmetic processing apparatus according to the embodiment.

FIG. 19 is an explanatory diagram schematically illustrating an example of data analysis processing in the arithmetic processing apparatus according to the embodiment.

FIG. 20 is a block diagram schematically illustrating an example of a hardware configuration of the arithmetic processing apparatus according to the embodiment.

### DESCRIPTION OF EMBODIMENTS

Hereinafter, (a) preferred embodiment(s) of the present disclosure will be described in detail with reference to the appended drawings. In this specification and the appended drawings, structural elements that have substantially the same function and structure are denoted with the same reference numerals, and repeated explanation of these structural elements is omitted.

Hereinafter, a description will be given in the following order.

1. Investigation by inventors

2. Embodiments

2.1. Imaging apparatus

2.2. Examples of configuration of imaging apparatus

2.3. Configuration of arithmetic processing apparatus

2.4. Imaging method

2.5. Hardware configuration

(Investigation by the Inventors)

The inventors investigated actions that doctors desire in the medical field, including in methods of identifying a sentinel lymph node as disclosed in Non-Patent Literature 1. As a result, the inventors found that actions demanded by doctors in the medical field relate to various inspection and analysis operations and medical treatment performed on parts (i.e., affected areas) of biotissue (e.g., various organs and the like) corresponding to observation targets while doctors check the biotissue with the naked eye (i.e., using light within a visible light band). Particularly, such demands grow when various inspection and analysis operations are performed, for example, using light that is not visible to doctors with the naked eye (i.e., light outside of the visible light band) as in a method using fluorescence, such as the aforementioned ICG, a method using optical ultrasonic waves, optical coherence tomography (OCT) and the like.

However, to realize both observation of biotissue in the visible light band by a doctor or the like and the aforementioned inspection and analysis operations using light, it is important to mount two units for realizing the respective functions in medical observation equipment and therefore the equipment tends to increase in size. When an object on which the units are mounted is originally large medical observation equipment, for example, a microscope for ophthalmic operation, the equipment further increases in size, but mounting the units for performing inspection and analysis operations in the equipment does not cause a particularly serious problem. However, when the aforementioned units for performing inspection and analysis operations are mounted in originally small medical observation equipment, for example, various endoscopes, arthroscopes and the like, equipment size increase is undesirable because it is important for doctors and the like to be able to perform predetermined inspection and analysis operations while holding such an endoscope, an arthroscope or the like.

With respect to the aforementioned needs, the inventors found from examination results that it is possible to realize both observation of biotissue in the visible light band and the aforementioned various inspection and analysis operations using light even when the size of medical observation equipment, such as various endoscopes and arthroscopes, is limited if an imaging apparatus used when an imaging target such as the biotissue or the like is imaged can be miniaturized.

Accordingly, as a result of further investigation based on the aforementioned consideration, the inventors learned that space saving can be achieved and equipment size can be reduced by causing optical paths for realizing an operation of observing biotissue in the visible light band and the aforementioned various inspection and analysis operations using light to be coaxial.

The inventors devised an imaging apparatus according to an embodiment of the present disclosure on the basis of such consideration, as will be described in detail below.

### EMBODIMENTS

<Imaging Apparatus>

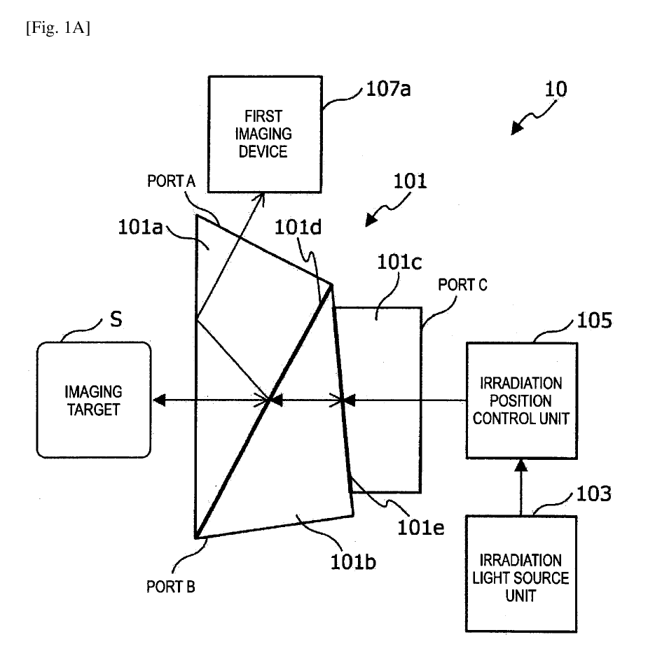

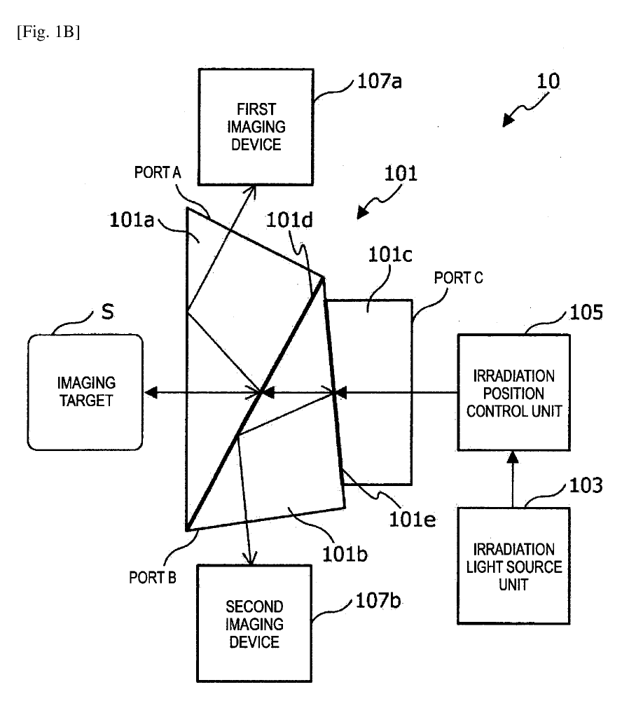

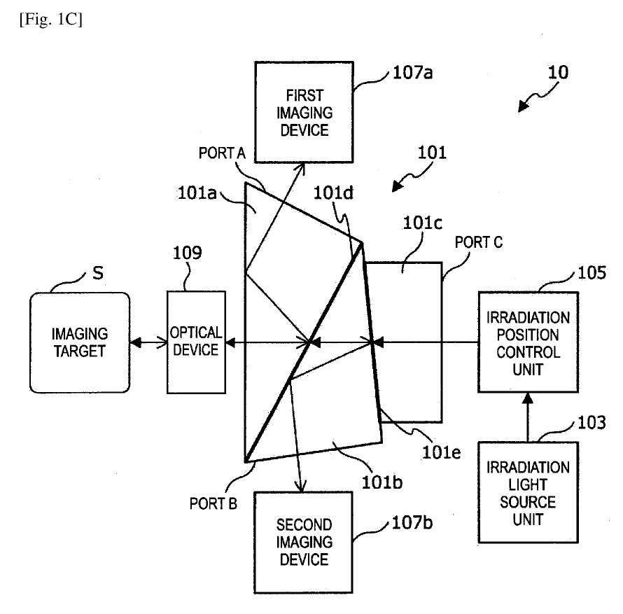

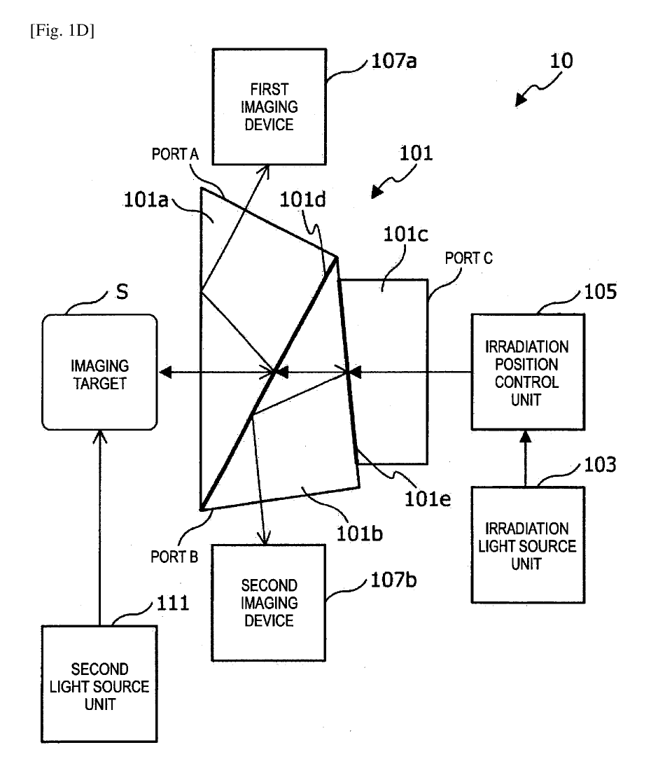

First of all, a configuration of an imaging apparatus according to an embodiment of the present disclosure will be described in detail below with reference to FIGS. 1A to 4.

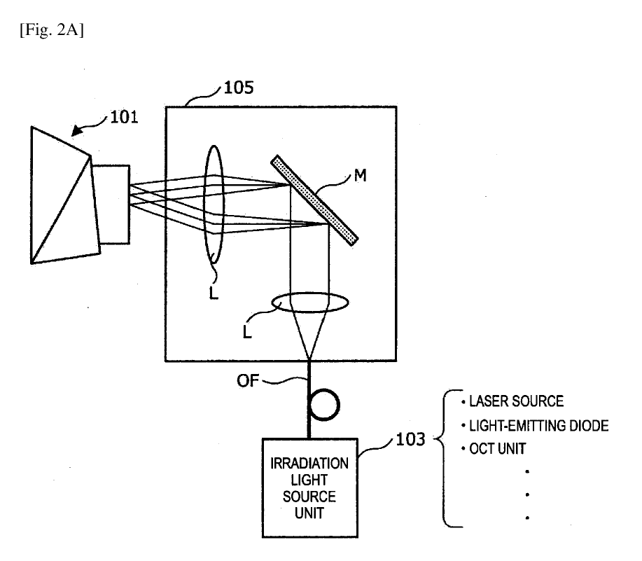

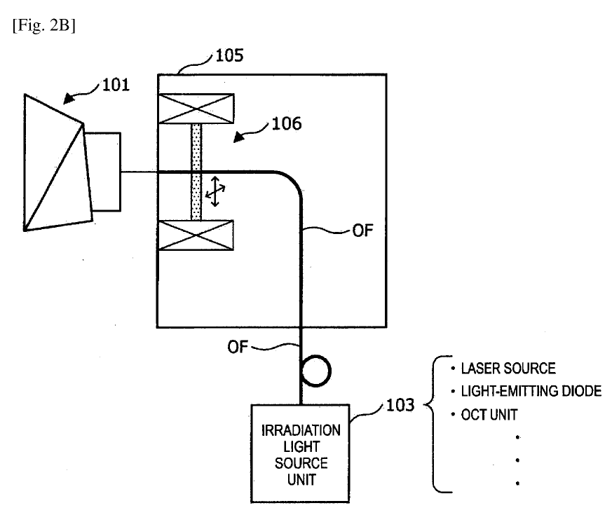

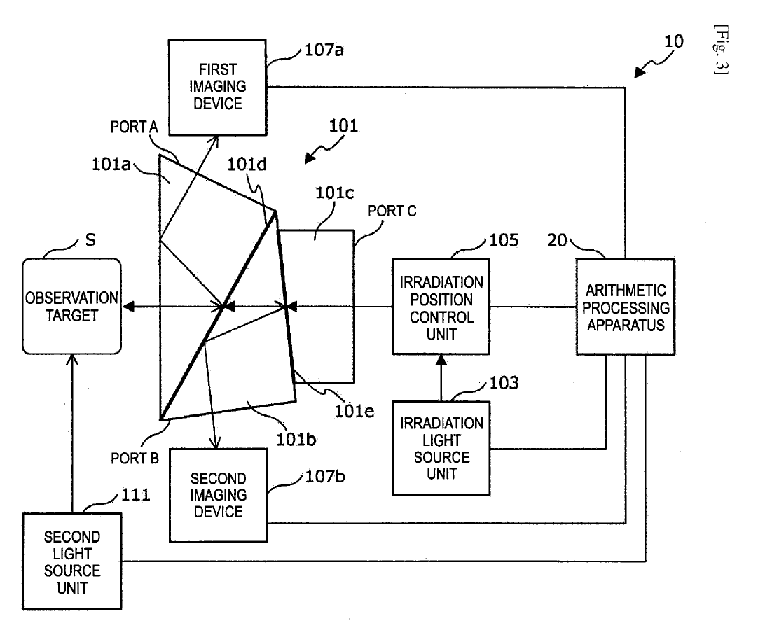

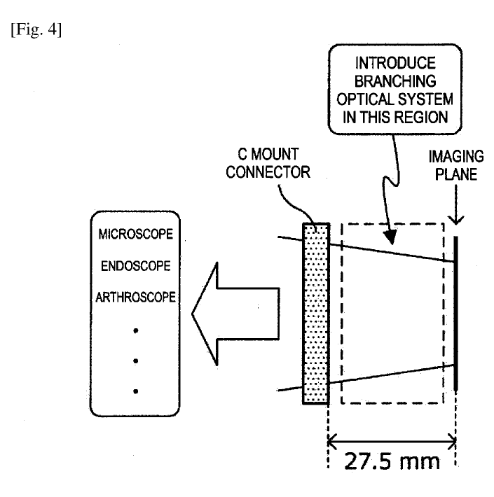

FIGS. 1A and 3 are explanatory diagrams schematically illustrating examples of the configuration of the imaging apparatus according to the present embodiment. FIGS. 2A and 2B are explanatory diagrams schematically illustrating examples of a configuration of an irradiation position control unit included in the imaging apparatus according to the present embodiment. FIG. 4 is an explanatory diagram of a branching optical system included in the imaging apparatus according to the present embodiment.

The imaging apparatus according to the present embodiment is an apparatus for imaging an imaging target (e.g., biotissue or the like) to generate various captured images including a captured image of the imaging target in the visible light band. The imaging apparatus includes a branching optical system that coaxially branches incident light into at least three different types of optical paths, an irradiation light source unit that emits light having a predetermined wavelength to the imaging target, an irradiation position control unit that controls an irradiation position of irradiation light emitted from the irradiation light source unit on the imaging target, and at least one imaging device that images light from the imaging target.

An example in which the aforementioned branching optical system coaxially branches incident light into three types of optical paths will be described in detail below.

As schematically illustrated in FIGS. 1A and 1B, a spectral prism having three or more types of optical prisms, which are joined to one another, can be used as the branching optical system 101 according to the present embodiment. In the example illustrated in FIGS. 1A and 1B, the branching optical system 101 includes a first prism 101a, a second prism 101b and a third prism 101c, which are sequentially disposed from the side close to an imaging target S, and these three types of optical prisms are joined to one another.

In the branching optical system 101 as illustrated in FIGS. 1A and 1B, there is one optical path at the side corresponding to the imaging target S, whereas the optical path is branched into three types at the other side of the branching optical system 101. Hereinafter, the end of a branched optical path in the first prism 101a is referred to as port A and, likewise, the end of a branched optical path in the second prism 101b is referred to as port B and the end of a branched optical path in the third prism 101c is referred to as port C.

In the past, such a branching optical system was used to coaxially branch light input from the imaging target to three optical paths or to combine light input from the respective ports and emit the combined light to the imaging target. That is, in the past, light propagated only in one direction such as left to right or right to left in FIG. 1A in the branching optical system.

In the branching optical system 101 according to the present embodiment, however, at least parts of three or more types of optical paths are used as an optical path for guiding irradiation light, which will be described below, to the imaging target S and an optical path for guiding light from the imaging target S. Accordingly, it is possible to apply the irradiation light having a controlled irradiation position, which will be described below, to the imaging target S through a first optical path in the branching optical system 101 and to guide the light from the imaging target S to the at least one imaging device through an optical path other than the first optical path in the branching optical system 101.

To realize propagation of light in two directions in the aforementioned branching optical system 101, the joint surface 101d between the first prism 101a and the second prism 101b and the joint surface 101e between the second prism 101b and the third prism 101c serve as at least one of a beam splitter (BS), a polarizing beam splitter (PBS) and a wavelength selective filter in the branching optical system 101. Accordingly, light beams propagating through the three optical paths can be distinguished.

While positions at which three types of optical devices or the like can be installed, such as port A to port C, are present in the branching optical system 101, as schematically illustrated in FIG. 1A, an irradiation position control unit 105 which will be described below is provided at any of the port A to the port C and the imaging device 107 is provided at at least one of the remaining ports in the imaging apparatus 10 according to the present embodiment. In the example illustrated in FIG. 1A, the irradiation position control unit 105 is provided at the port C of the branching optical system 101 and the irradiation light source unit 103 is provided above the irradiation position control unit 105. In addition, a first imaging device 107a is provided at the port A of the branching optical system 101.

Although the port B corresponding to the second prism 101b is not used in the example illustrated in FIG. 1A, a second imaging device 107b may be provided at the port B as illustrated in FIG. 1B.

Further, a relationship between the ports of the branching optical system 101 and parts provided thereat is not limited to the examples shown in FIGS. 1A and 1B, and the irradiation position control unit 105 and the imaging device 107 may be installed at positions of any ports of the branching optical system 101.

Installation of optical devices at positions of ports corresponding thereto and functions of the two joint surfaces in the branching optical system 101 will be described in more detail below.

When the branching optical system 101 is used, an observation function using the visible light band and inspection and analysis functions can be implemented within a reduced space and medical observation equipment can be miniaturized. Furthermore, because the optical paths are integrated and are coaxial in the branching optical system 101, it is easy to perform position adjustment between optical paths and it is possible to apply irradiation light to any position of the imaging target while performing observation.

The irradiation light source unit 103 included in the imaging apparatus 10 according to the present embodiment is a part which emits light having a predetermined wavelength to the imaging target S. Light emitted from the irradiation light source unit 103 is not particularly limited, and a visible laser source for indicating the position of an imaging target may be provided (simply referred to hereinafter as “position-indicating laser source”). In addition to the position-indicating laser source, laser sources having various wavelength bands including the visible laser source and various low coherence light sources such as a light-emitting diode may be provided. Further, a light source for a specific purpose (e.g., a time-of-flight (TOF) measurement light source for performing a TOF method) may be provided as the irradiation light source unit 103. Moreover, an optical coherence tomography (OCT) unit which acquires an optical tomographic image of the imaging target S by applying irradiation light having infrared wavelengths to the imaging target S and detecting reflected light of the irradiation light having the infrared wavelengths from the imaging target S may be provided as the irradiation light source unit 103.

The irradiation light emitted from the irradiation light source unit 103 may be used for inspection and analysis of biotissue or the like corresponding to the imaging target and for treatment of the biotissue or the like corresponding to the imaging target, but the use thereof is not limited.

The irradiation light emitted from the irradiation light source unit 103 is guided to the irradiation position control unit 105. While a method of guiding the irradiation light from the irradiation light source unit 103 to the irradiation position control unit 105 is not particularly limited and may be realized using various known lenses or mirrors, it is desirable to use various optical fibers in consideration of handling and safety of the irradiation light.

The irradiation position control unit 105 controls the irradiation position of the irradiation light emitted from the irradiation light source unit 103 on the imaging target S. The irradiation light can be applied to a desired point of the imaging target S by controlling the irradiation position of the irradiation light through the irradiation position control unit 105. As a result, a desired position of the imaging target S can be scanned with the irradiation light in the imaging apparatus 10 according to the present embodiment. In other words, the irradiation position control unit 105 according to the present embodiment is an optical system functioning as a scanning optical system and the whole irradiation position control unit 105 serves as a scanner.

Although the irradiation position control unit 105 is not limited, the irradiation position of the irradiation light may be controlled by combining a mirror M and two types of lenses L, installing the mirror M at a position conjugated with respect to the positions of the ports of the branching optical system 101 and then operating the mirror M, for example, as shown in FIG. 2A. As the operation mirror, for example, a known mirror such as a galvanomirror or a microelectro-mechanical system (MEMS) mirror may be used. When the galvanomirror is used, highly accurate scanning can be realized but there is a possibility of the irradiation position control unit 105 increasing in size. Accordingly, it is desirable to use the MEMS mirror as the operation mirror when the imaging apparatus 10 according to the present embodiment is mounted in medical observation equipment of which miniaturization is necessary, such as an endoscope and an arthroscope.

In addition, as shown in FIG. 2B, a scanning unit which performs scanning of irradiation light by varying the position of the exit end of an optical fiber OF for guiding the irradiation light by installing a control mechanism 106 capable of controlling the position of the exit end of the optical fiber OF may be realized as the irradiation position control unit 105, for example. The control mechanism 106 is not particularly limited but may be realized using various motors, actuators or the like. Here, a structure in which a ball lens or a cylindrical lens having a coaxially varying refractive index, which is commonly called a SELFOC lens, is provided at the exit end of the optical fiber OF to control the emission angle of light emitted from the optical fiber OF or focus the light may be employed.

The imaging device 107, which images light from the imaging target S, can detect light intensity distribution at the position thereof, and a known imaging device, for example, any of various charge-coupled device (CCD) image sensors, complementary MOS (CMOS) image sensors or the like, can be used thereas. In the imaging apparatus 10 according to the present embodiment, a wavelength band of light sensed by the imaging device is not limited and a combination of imaging devices may be determined depending on a related wavelength band of light.

For example, only visible light imaging devices may be used when only light belonging to the visible light band is of concern, infrared light imaging devices may be used when light belonging to the infrared light band is of concern, and both the visible light imaging devices and the infrared light imaging devices may be used when both light belonging to the visible light band and light belonging to the infrared light band are of concern. Further, if fluorescence belonging to a specific wavelength band is of concern, imaging devices having sensitivity to the wavelength band including the fluorescence may be appropriately used.

Furthermore, an imaging device for a specific purpose (e.g., a time-of-flight (TOF) measurement imaging device for performing a TOF method) may be provided as the imaging device 107 according to the present embodiment.

The imaging apparatus 10 according to the present embodiment may include, for example, various optical devices 109, such as a field lens and a quarter wave plate, between the branching optical system 101 and the imaging target S, as illustrated in FIG. 1C, in addition to the aforementioned branching optical system 101, the irradiation light source unit 103, the irradiation position control unit 105 and the imaging device 107. For example, the irradiation light may be applied to the imaging target S more uniformly when the optical device 109 such as a field lens is provided. If the optical device 109 such as a quarter wave plate is provided, more complicated light splitting can be realized in the branching optical system 101.

The imaging apparatus 10 according to the present embodiment may include, for example, a second light source unit 111, as illustrated in FIG. 1D, in addition to the aforementioned branching optical system 101, the irradiation light source unit 103, the irradiation position control unit 105 and the imaging device 107. The second light source unit 111 emits second light different from the irradiation light emitted from the irradiation light source unit 103, and the second light is applied to the imaging target S without passing through the branching optical system 101.

When the second light source unit 111 is provided, it may be possible to apply excitation light having a predetermined wavelength to biotissue corresponding to an imaging target or various chemical materials included in the biotissue to change the biotissue corresponding to the imaging target or the various chemical materials included in the biotissue into a desired state in inspection and analysis operations, photo-dynamic diagnosis (PDD) and the like using fluorescence such as the ICG method, for example. When fluorescence is observed, an EM filter for absorbing the wavelength of excitation light for exciting fluorescence may be provided between an imaging device and a prism such that the excitation light is not input to the imaging device, thereby improving signal quality of fluorescent images.

Of course, the imaging apparatus 10 according to the present embodiment may include both the optical device 109, as illustrated in FIG. 1C, and the second light source unit 111, as illustrated in FIG. 1D, in addition to the aforementioned branching optical system 101, the irradiation light source unit 103, the irradiation position control unit 105 and the imaging device 107.

It is desirable that the aforementioned imaging apparatus 10 according to the present embodiment further include an arithmetic processing apparatus 20, as shown in FIG. 3, for example. The arithmetic processing apparatus 20 collectively controls the irradiation light source unit 103, the irradiation position control unit 105 and the at least one imaging device 107 and acquires image data of a captured image generated in the at least one imaging device 107. Further, when the imaging apparatus 10 according to the present embodiment further includes the second light source unit 111, as shown in FIG. 3, the arithmetic processing apparatus 20 may further control the second light source unit 111. Functions of the arithmetic processing apparatus 20 will be described in more detail below.

Since the imaging apparatus 10 according to the present embodiment, as illustrated in FIGS. 1A to 3, includes the branching optical system 101 that demands a reduced space, as described above, the imaging apparatus 10 may be mounted in medical observation equipment having a C mount attached thereto or medical observation equipment having a C mount adaptor attached thereto. Although the C mount has a size in which a distance between a connector part and an imaging plane is designated as 5 mm, as schematically illustrated in FIG. 4, the branching optical system 101 according to the present embodiment can be applied even to the limited area of 27.5 mm. Accordingly, the imaging apparatus 10 according to the present embodiment can also be mounted in small medical observation equipment gripped by a user for observation, such as the endoscope, arthroscope and the like.

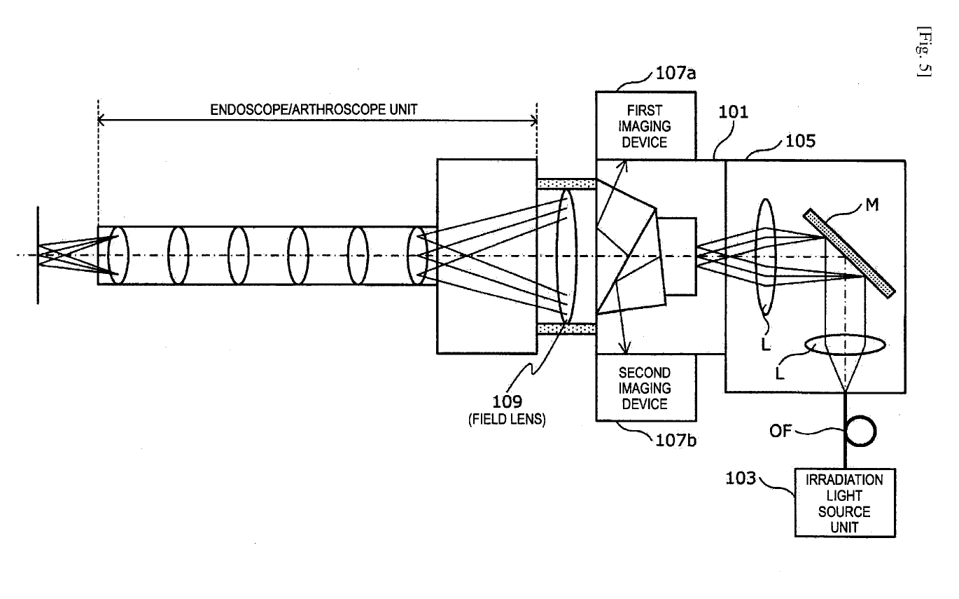

FIG. 5 schematically illustrates the optical system of the imaging apparatus 10 according to the present embodiment when the imaging apparatus 10 is mounted in an endoscope/arthroscope unit. In this case, a field lens is preferably provided as the optical device 109 between the branching optical system 101 and the endoscope/arthroscope unit, as illustrated in FIG. 5. Accordingly, the irradiation light emitted from the irradiation light source unit 103 may be uniformly guided to the tip of the endoscope/arthroscope unit. An image of biotissue or the like acquired by the endoscope/arthroscope unit is branched by the branching optical system 101 and imaged by the first imaging device 107a and the second imaging device 107b. In the branching optical system 101 according to the present embodiment, it may be possible to selectively branch the image of the biotissue or the like acquired by the endoscope/arthroscope unit by causing the joint surfaces of the optical prisms to have specific functions. Accordingly, it may also be possible to intentionally change the images of the biotissue or the like formed by the first imaging device 107a and the second imaging device 107b. Therefore, an image in the visible light band may be formed by one imaging device whereas an image in the infrared light band may be formed by the other imaging device.

The imaging apparatus 10 according to the present embodiment has been described in detail with reference to FIGS. 1A to 5.

<Examples of Configuration of Imaging Apparatus>

Examples of the configuration of the aforementioned imaging apparatus 10 will be described in detail below.

As described above, the imaging apparatus 10 according to the present embodiment can realize various functions when the irradiation light source unit 103 and the imaging device 107 provided at respective ports and functions assigned to the joint surfaces are appropriately selected.

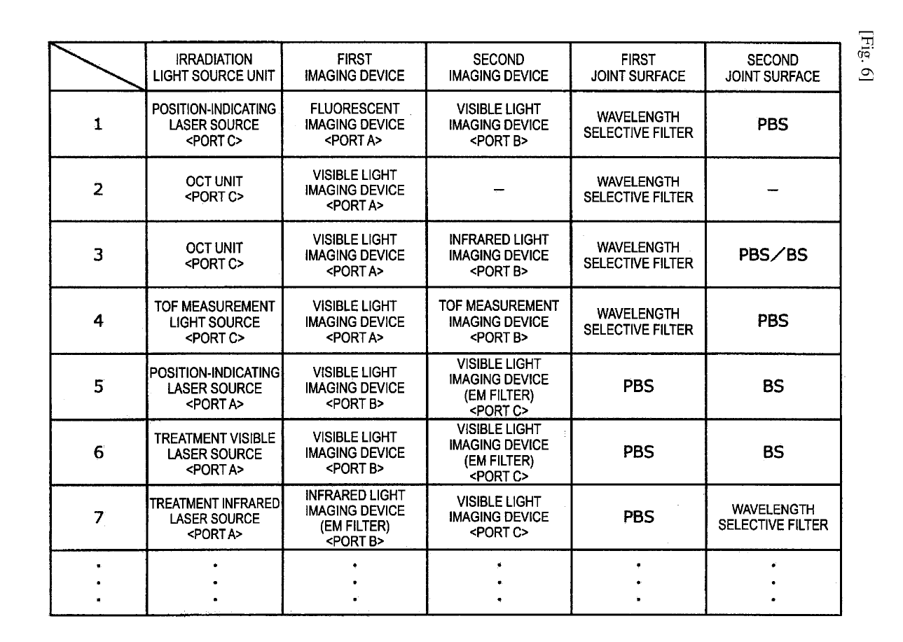

FIG. 6 illustrates examples of functions that can be realized in the imaging apparatus 10 according to the present embodiment. The examples shown in FIG. 6 are merely exemplary and functions that can be realized in the imaging apparatus 10 according to the present embodiment are not limited thereto.

<<Example of Configuration of No. 1 of FIG. 6>>

In the imaging apparatus 10 according to the present embodiment, for example, a position-indicating visible laser source may be provided as the irradiation light source unit 103, a fluorescent imaging device capable of performing fluorescent imaging and a visible light imaging device may be provided as the imaging device 107, a wavelength selective filter may be provided at the first joint surface 101d, and the second joint surface 101e may serve as a polarizing beam splitter (PBS) (No. 1 of FIG. 6). Accordingly, it may be possible to recognize fluorescence that is not visible in the visible light band, such as ICG, using a captured image from the fluorescent imaging device and to indicate a fluorescence emission region in biotissue by using a position-indicating laser like a laser pointer.

Although a fluorescence method using ICG is used to identify a sentinel lymph node in operations for breast cancer, as described above, the rate of introduction of such a method is low. This is because doctors who are operators can only view an image of the sentinel lymph node observed through an imaging device for infrared light only through a monitor and are not able to recognize the position of the lymph node unless they avert their eyes from the field of operations because fluorescence from ICG has a wavelength that is not observed with the naked eye. When a doctor performs an operation using a hard type endoscope or a soft type endoscope, the doctor who is an operator can easily check a resection region by superposing an ICG observation image on a monitor displaying an endoscope image. However, in the case of operations to open the stomach/chest without using an imaging device for resection, such as operations for breast cancer, a doctor who is an operator has to avert his or her eyes from the field of operations in order to pay attention to a captured image (infrared captured image) from an imaging device for infrared light and thus is in danger of misrecognizing a related position in the infrared captured image in the field of operations. Therefore, to widely use sentinel lymph node biopsy according to the ICG method, it is important to realize a method through which doctors who are operators can detect the position of the sentinel lymph node without averting their eyes from the field of operations even in laparotomy.

In such a situation, projection of observation images of ICG and the like or images of CT and the like using a projector in the field of operations is investigated. However, there are problems that the field of operations is not a plane unlike a screen and thus has an unfocused region, a projection part is enlarged and thus a rack larger than a rack that accommodates only a camera is necessary to accommodate the projection part, and so on.

However, when the imaging apparatus 10 according to the present embodiment is installed above the field of operations, a fluorescence emission form can be imaged by the imaging apparatus 10 and a doctor who is an operator can easily specify a fluorescence emission region in the field of operations by emitting a position-indicating visible laser from the imaging apparatus 10 to the field of operations. Furthermore, the irradiation position of the position-indicating visible laser is scanned by the irradiation position control unit 105 included in the imaging apparatus 10 according to the present embodiment, and thus the position can be designated even in the field of operations, which is not a plane, without blurring.

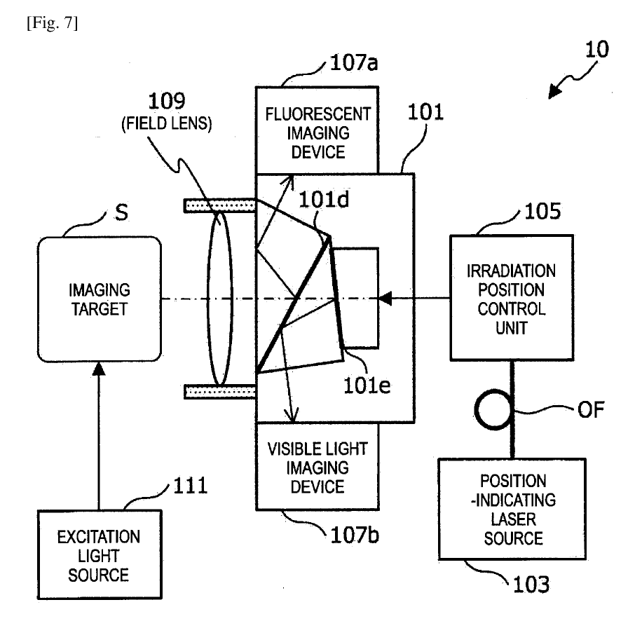

The optical system in this configuration example is schematically illustrated in FIG. 7. In the imaging apparatus 10 according to the present configuration example, the irradiation position control unit 105 is provided at the port C of the branching optical system 101, a fluorescent imaging device is provided as the first imaging device 107a at the port A of the branching optical system 101, and a visible light imaging device is provided as the second imaging device 107b at the port B of the branching optical system 101. In addition, a filter configured to transmit visible light while reflecting infrared light (e.g., a filter which passes light having a wavelength of 700 nm or lower and reflects light having a wavelength of higher than 700) is provided as the wavelength selective filter at the first joint surface 101d, and a PBS is provided at the second joint surface 101e. As the irradiation light source unit 103, a visible laser source such as a green laser source is provided as the position-indicating laser source, for example. To radiate the position-indicating visible laser beam more uniformly, a field lens is provided as the optical device 109 between the branching optical system 101 and the imaging target S.

In the present configuration example, in order to excite a fluorescent material such as ICG, an excitation light source adapted to the excitation wavelength of the used fluorescent material is provided as the second light source unit 111 and excitation light is emitted without passing through the branching optical system 101.

The imaging apparatus illustrated in FIG. 7 enables observation of an image of visible light or infrared light and spot emission of a visible laser beam to biotissue or the like corresponding to the imaging target. In addition, polarization of the irradiation light from the irradiation light source unit 103 is controlled such that the irradiation light can pass through the PBS provided at the second joint surface 101e, and thus the irradiation light can be radiated to the imaging target with high efficiency and generation of stray light in the branching optical system 101 can be sufficiently restricted.

In identification of the sentinel lymph node using ICG, an operating surgeon has to view images displayed on a monitor in general because the operating surgeon is not able to observe infrared light observation images with his or her eyes, as described above. However, in the imaging apparatus 10 of the present configuration example, an assistant may check the infrared light observation images through the monitor and perform a predetermined user operation for the imaging apparatus 10 (more specifically, the arithmetic processing apparatus 20) to control the irradiation position control unit 105. Accordingly, it is possible to irradiate the field of operations with a visible laser pointer and indicate a fluorescence emission region to the operating surgeon. While a method of indicating the fluorescence emission region is not particularly limited, it is desirable to control the irradiation position control unit 105 to trace a position corresponding to the form of the fluorescence emission region. Accordingly, the operating surgeon can recognize the position of the sentinel lymph node without averting his or her gaze from the field of operations.

In a related method, control may be automated such that the laser pointer indicates a region having a high luminance value in an infrared light observation image even if the assistant does not control the irradiation position control unit 105 according to user operation.

Here, the size of the sentinel lymph node is generally several mm (about 3 mm to 10 mm). When the observation field of the imaging apparatus 10 is about 50 cm, attention is paid to a necessary resolution (necessary spot size) of the laser pointer. Here, it is assumed that an image captured by the imaging apparatus 10 is a high vision image having 1920×1080 pixels and the optical system supports this resolution. In this case, if a general lens having a pupil diameter of 6 mm is used, it is important to input a beam with a diameter of 6 mm to the lens when an irradiation area is irradiated with a spot diameter of about 0.25 mm. Here, when a relay lens is not used in a MEMS mirror or the like, a beam diameter is about 0.6 mm when the MEMS mirror is installed at an angle of 45 degrees according to the method illustrated in FIG. 2A because a diameter of approximately 1 mm corresponds to the MEMS mirror size. When the beam with a diameter of approximately 0.6 mm is input to the lens with a pupil diameter of 6 mm, although the resolution decreases by a factor of ten because the beam diameter becomes approximately 1/10, the beam is still focused with a spot diameter of about 2.5 mm. However, since the size of the sentinel lymph node is several mm (about 3 mm to 10 mm) as described above, the irradiation spot size of 2.5 mm is smaller than the size of the sentinel lymph node. Accordingly, even the irradiation position control unit 105 using a small MEMS scan mirror instead of a large galvanomirror can indicate a fluorescence emission region to a doctor. Furthermore, because the galvanomirror may not be used as the irradiation position control unit 105, the entire imaging apparatus 10 can be configured to be small and lightweight. The fact that the imaging apparatus 10 is lightweight means that an arm supporting the imaging apparatus 10 can also be lightweight, thus decreasing costs and saving space in an area in which size is limited, such as an operating room.

<<Example of Configuration of No. 2 and No. 3 of FIG. 6>>

The imaging apparatus 10 according to the present embodiment may realize a function of performing OCT imaging while biotissue corresponding to an imaging target is observed with the naked eye (No. 2 and No. 3 of FIG. 6).

In Japan, not only is the MRI supply rate in hospitals having a large number of beds high but there are many facilities which have imaging equipment such as MRI and CT and perform image diagnosis using this equipment in outpatient clinics, and thus even patients of private orthopedic offices have the opportunity for MRI diagnosis. However, since the MRI supply rate is low in countries other than Japan, patients who have diseases that would be diagnosed according to MRI in Japan have fewer opportunities for MRI diagnosis in other countries. That is, a patient having a disease of cartilage such as a knee joint, more specifically, a patient of a disease such as a meniscus injury, undergoes medical diagnosis according to MRI and then gets surgical treatment using an arthroscope if necessary in Japan. In the United States and other countries where the MRI supply rate is low, however, a patient of a meniscus injury that is not able to be detected by CT undergoes arthroscopy without MRI diagnosis.

However, when the arthroscope is applied to patients of a meniscus injury that can be diagnosed according to MRI, some patients are not able to be diagnosed because the arthroscope has no fluoroscopic function. For example, a meniscus injury includes breaking and cracking, and the diagnosis capability of an arthroscope alone does not compare to that of MRI. While there is optical coherence tomography (OCT) as a method of observing the tissue of the human body using wavelengths having high fluoroscopic property, it is difficult to maintain the size of the arthroscope such that a doctor can hold the arthroscope with his or her hand while mounting an OCT unit on the arthroscope having a diameter of about 4 mm.

However, when the imaging apparatus 10 having a reduced size according to the present embodiment is attached to the C mount connector of the arthroscope, endoscope or the like, it is possible to realize an arthroscope and an endoscope having the OCT function.

As a related configuration example, a configuration of No. 2 of FIG. 6 is exemplified.

In this configuration, the irradiation position control unit 105 is provided at the port C of the branching optical system 101 and a visible light imaging device is provided as the first imaging device 107a at the port A of the branching optical system 101. In addition, a filter which reflects visible light and transmits infrared light (e.g., a filter which reflects light having a wavelength of 700 nm or lower and transmits light having a wavelength higher than 700 nm) as the wavelength selective filter is provided at the first joint surface 101d. Further, an OCT unit is mounted as the irradiation light source unit 103. It is desirable that a field lens be provided as the optical device 109 between the branching optical system 101 and the imaging target S in order to radiate infrared light from the OCT unit more uniformly.

In this case, observation light in the visible light band is imaged by the visible light imaging device because visible light is reflected by the first joint surface 101d, realizing image observation in the visible light band. Furthermore, when a doctor performs observation using a visible light observation image generated by the visible light imaging device and specifies a region from which he or she wants to acquire OCT information, infrared light having a wavelength of 1300 nm, for example, emitted from the OCT unit is focused as a beam at the position of the port C corresponding to the region by the irradiation position control unit 105. Then, the infrared light passes through the second joint surface 101e and the first joint surface 101d and is applied to a predetermined portion of biotissue through the arthroscope. In addition, reflected light of irradiation light from the OCT unit passes through the C mount, the first joint surface 101d and the second joint surface 101e through the arthroscope and then is finally analyzed by the OCT unit.

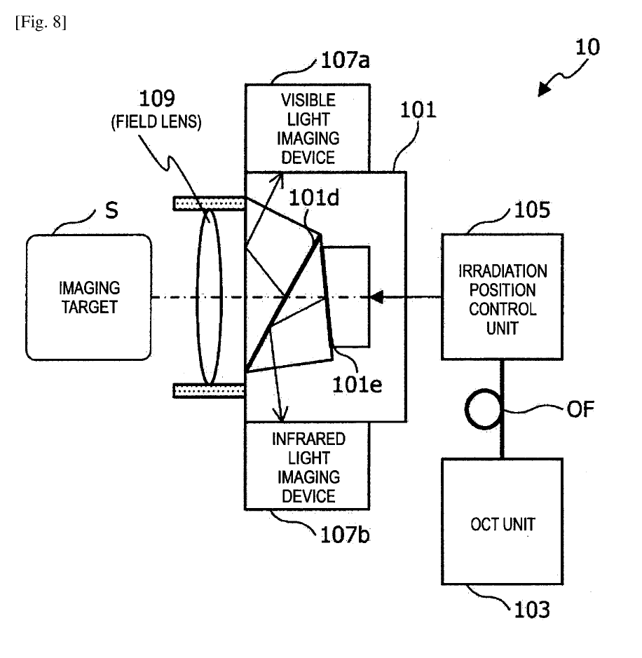

While the wavelength selective filter is provided at the first joint surface 101d and the second joint surface 101e does not have a specific reflection function in the configuration example of No. 2, an observation image of infrared light emitted from the OCT unit may be obtained by employing a configuration as represented by No. 3 of FIG. 6, for example. The optical system in this configuration is schematically illustrated in FIG. 8.

In this case, an infrared light imaging device is provided as the second imaging device 107b at the port B of the branching optical system 101, which is not used in the configuration of No. 2, and the second joint surface 101e serves as a polarizing beam splitter (PBS) or a beam splitter (BS). Accordingly, reflected light of irradiation light from the OCT unit arrives at the second joint surface 101e through the arthroscope and thus both the infrared light imaging device and the OCT unit form images.

In the configuration example illustrated in FIG. 8, it may be possible to generate an integrated image of a visible light observation image and an infrared light observation image very easily by previously performing position alignment between the infrared light imaging device and the visible light imaging device.

In addition, light from the OCT unit is applied to the imaging target with high efficiency since an appropriate PBS is provided at the second joint surface 101e and polarization of infrared light from the OCT unit is controlled such that the infrared light passes through the second joint surface 101e. In the case of OCT measurement of the imaging target having no change in polarization, it is possible to analyze reflected light with OCT as it is and to realize functions of an IR camera.

It is possible to acquire a visible light image in medical observation equipment including a camera mount, C mount, such as a microscope, an arthroscope or an endoscope and to perform laser analysis such as simple OCT according to the aforementioned configuration example.

When the configuration example is applied to the arthroscope, it is proven that resolution of 1 pixel at which high vision observation is performed is not necessary but slightly lower resolution is sufficient for OCT of the arthroscope. Even in this case, therefore, it is desirable to decrease the imaging apparatus in size by employing a scanning mechanism using a MEMS mirror as the irradiation position control unit 105. Specifically, when the observation field is 40 nm, although 1 pixel of a high vision image corresponds to 20.8 micro meter, related resolution has a value less than the diameter of a human hair. In the meantime, since resolution in the range of 0.2 mm to 0.3 mm is necessary when meniscus diagnosis is performed through MRI and thus MRI diagnosis can be performed with approximately 15 times the resolution of optical cameras, it is possible to realize resolution capable of achieving MRI diagnosis even when a beam having a diameter corresponding to 1/15 of the pupil diameter is input. Accordingly, it is possible to achieve resolution equal to that of MRI even when the small irradiation position control unit 105 using a MEMS mirror is used instead of the large irradiation position control unit 105 using a galvanomirror. Here, it may also be possible to obtain resolution with high accuracy even with the MEMS mirror by using a relay lens optical system because a beam diameter can be increased using a relay lens even when a small scan mirror is employed. However, in order to achieve resolution with higher accuracy using the MEMS mirror, there is the possibility of the optical system becoming relatively large because of the space that the relay lens occupies.

<<Example of Configuration of No. 4 of FIG. 6>>

The imaging apparatus 10 according to the present embodiment may realize a function of measuring a distance to an imaging target (TOF measurement function) while biotissue corresponding to the imaging target is observed with the naked eye (No. 4 of FIG. 6).

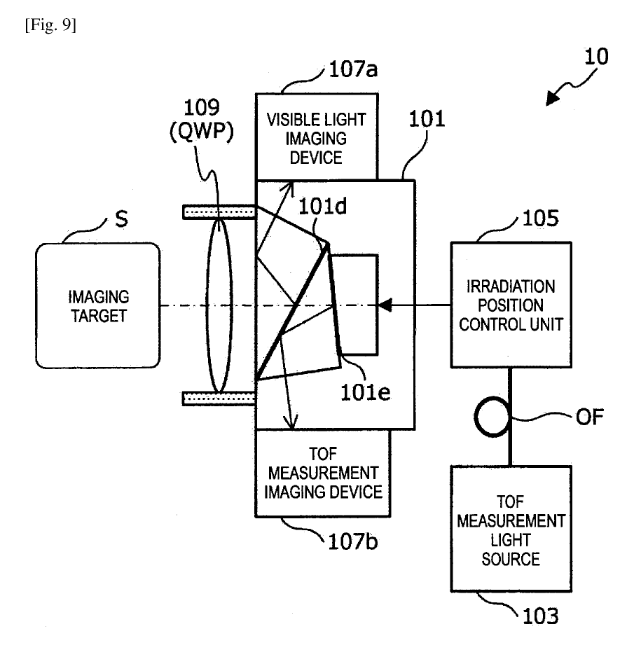

The optical system in the related configuration example is schematically illustrated in FIG. 9. In the imaging apparatus 10 of the present configuration example, the irradiation position control unit 105 is provided at the port C of the branching optical system 101, a visible light imaging device is provided as the first imaging device 107a at the port A of the branching optical system 101, and a TOF measurement imaging device (e.g., a TOF camera capable of measuring TOF or the like) is provided as the second imaging device 107b at the port B of the branching optical system 101. In addition, a filter which reflects visible light and transmits infrared light (e.g., a filter which reflects light having a wavelength of 700 nm or lower and transmits light having a wavelength of higher than 700) is provided as the wavelength selective filter at the first joint surface 101d, and a polarizing beam splitter (PBS) is provided at the second joint surface 101e. Further, a TOF measurement light source for TOF measurement is provided as the irradiation light source unit 103. A quarter wave plate (QWP) is provided as the optical device 109 between the branching optical system 101 and the imaging target S.

In this case, TOF measurement light, which has been polarization controlled to be able to pass through the second joint surface 101e, passes through the second joint surface 101e and the first joint surface 101d to reach the quarter wave plate, and the polarization direction of the light is controlled to be a direction in which the light does not pass through the second joint surface 101e. Then, the TOF measurement light arrives at a point at which a distance will be measured and then is reflected and passes through the first joint surface 101d to reach the second joint surface 101e. The polarization of the reflected light is controlled such that the reflected light does not pass through the second joint surface 101e, and thus the reflected light is reflected at the second joint surface 101e and imaged by the TOF measurement imaging device. Light of the visible light band from the imaging target is reflected at the first joint surface 101d and imaged by the visible light imaging device. Accordingly, it is possible to realize the function of measuring the distance to the imaging target (TOF measurement function) while the biotissue corresponding to the imaging target is observed with the naked eye.

In the present configuration example, even if the resolution of the TOF measurement imaging device is insufficient, the resolution of the TOF measurement imaging device may be compensated according to scanning of the irradiation position of the TOF measurement light by the irradiation position control unit 105 because the irradiation position of the TOF measurement light is controlled by the irradiation position control unit 105.

<<Example of Configuration of No. 5 of FIG. 6>>

The imaging apparatus 10 according to the present embodiment may realize a function of performing photodynamic diagnosis (PDD) while biotissue corresponding to an imaging target is observed with the naked eye (No. 5 of FIG. 6). In PDD, while fluorescence of a predetermined wavelength is generated from portions corresponding to cancer, there is a problem that correlation between fluorescence emission regions and positions corresponding thereto in the field of operations are difficult to obtain. Accordingly, fluorescence emission regions in biotissue are indicated using a position-indicating laser like a laser pointer as in the configuration example of No. 1.

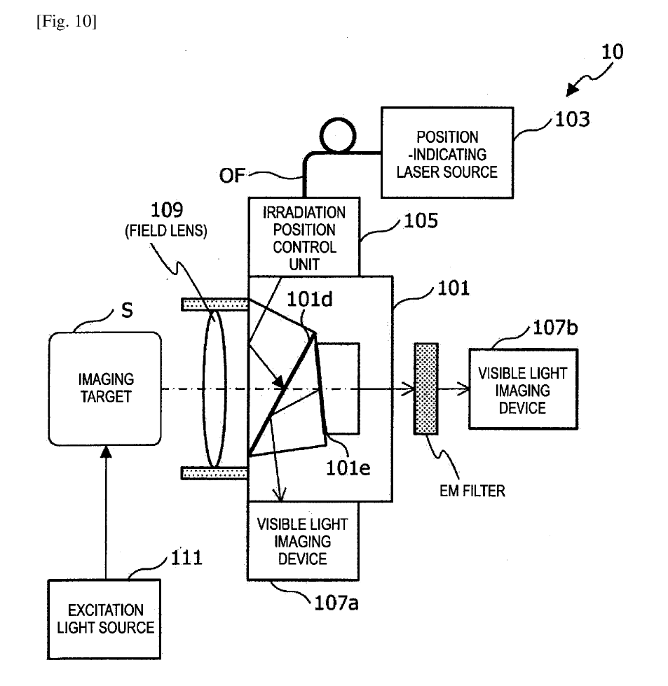

The optical system in the related configuration example is schematically illustrated in FIG. 10. In the imaging apparatus 10 of the present configuration example, the irradiation position control unit 105 is provided at the port A of the branching optical system 101, a visible light imaging device is provided as the first imaging device 107a at the port B of the branching optical system 101, and an EM filter that absorbs the wavelength of excitation light for exciting fluorescence and a visible light imaging device are provided at the port C of the branching optical system 101. In addition, a polarizing bean splitter (PBS) is provided at the first joint surface 101d and a beam splitter BS is provided at the second joint surface 101e. Further, a position-indicating laser source, for example, a visible laser source such as a green laser source, is provided as the irradiation light source unit 103. Moreover, a field lens is provided as the optical device 109 between the branching optical system 101 and the imaging target S in order to radiate the position-indicating visible laser beam more uniformly.

In the present configuration example, to excite a fluorescent material administered into the biotissue, an excitation light source adapted to the excitation wavelength of the used fluorescent material is provided as the second light source unit 111 and thus excitation light is radiated without passing through the branching optical system 101.

In the imaging apparatus illustrated in FIG. 10, visible light from the imaging target passes through the first joint surface 101d and then is branched into two beams by the second joint surface 101e, and one of the branched visible beams is imaged by the visible light imaging device 107a. Fluorescence generated according to excitation light from the excitation light source passes through the first joint surface 101d and the second joint surface 101e, and then is imaged by the visible light imaging device 107b after the excitation wavelength has been removed therefrom by the EM filter. A fluorescence emission region (i.e., a cancerous portion) can be specified through an observation image generated from the visible light imaging device 107b.

Further, polarization of the visible laser beam emitted from the position-indicating laser source is controlled such that the visible laser beam is reflected by the polarizing beam splitter at the first joint surface 101d, and the irradiation position of the visible laser is controlled by the irradiation position control unit 105 such that the visible laser is emitted to the fluorescence emission region. The visible laser beam of which the irradiation position has been controlled is reflected at the first joint surface 101d and applied to a position corresponding to the fluorescence emission region. Accordingly, the position-indicating visible laser is emitted from the imaging apparatus 10 to the field of operations, and thus a doctor who is an operator can easily specify fluorescence emission regions in the field of operations. Furthermore, since the irradiation position of the position-indicating visible laser is scanned by the irradiation position control unit 105, the position corresponding to the fluorescence emission region can be designated even in the field of operations which is not a plane without worrying about blurring.

<<Example of Configuration of No. 6 of FIG. 6>>

The imaging apparatus 10 according to the present embodiment may realize a function of performing photodynamic therapy (PDT) while biotissue corresponding to an imaging target is observed with the naked eye (No. 6 of FIG. 6).

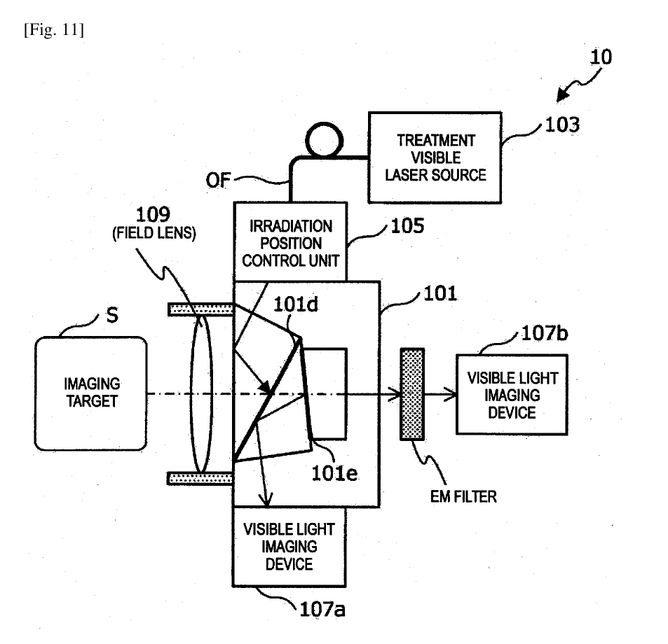

The optical system in the related configuration example is schematically illustrated in FIG. 11. In the imaging apparatus 10 of the present configuration example, the irradiation position control unit 105 is provided at the port A of the branching optical system 101, a visible light imaging device is provided as the first imaging device 107a at the port B of the branching optical system 101, and an EM filter that absorbs the wavelength of treatment visible light for exciting a light sensitive substance and a visible light imaging device are provided at the port C of the branching optical system 101. In addition, a polarizing bean splitter (PBS) is provided at the first joint surface 101d and a beam splitter BS is provided at the second joint surface 101e. Further, a treatment visible laser source for realizing treatment according to PDT by exciting the light sensitive substance captured in an affected area is provided as the irradiation light source unit 103. Moreover, a field lens is provided as the optical device 109 between the branching optical system 101 and the imaging target S in order to radiate the treatment visible laser beam more uniformly.

In the imaging apparatus illustrated in FIG. 11, visible light from the imaging target passes through the first joint surface 101d and then is branched into two beams by the second joint surface 101e, and one of the branched visible beams is imaged by the visible light imaging device 107a. The other visible beam is imaged by the visible light imaging device 107b after irradiation light from the treatment visible laser source is removed by the EM filter.

Further, polarization of the visible laser beam emitted from the treatment laser source is controlled such that the visible laser is reflected by the polarizing beam splitter at the first joint surface 101d, and the irradiation position of the visible laser is controlled by the irradiation position control unit 105 such that the visible laser is applied to a desired region. The visible laser beam of which irradiation position has been controlled is reflected at the first joint surface 101d and applied to the affected area for which PDT is performed.

<<Example of Configuration of No. 7 of FIG. 6>>

The imaging apparatus 10 according to the present embodiment may realize a function of performing photo-immunotherapy (PIT) while biotissue corresponding to an imaging target is observed with the naked eye (No. 7 of FIG. 6). PIT involves using a dye that bonds to only cancer cells and heating the dye by irradiating the dye with near-infrared light to extinguish cancer cells.

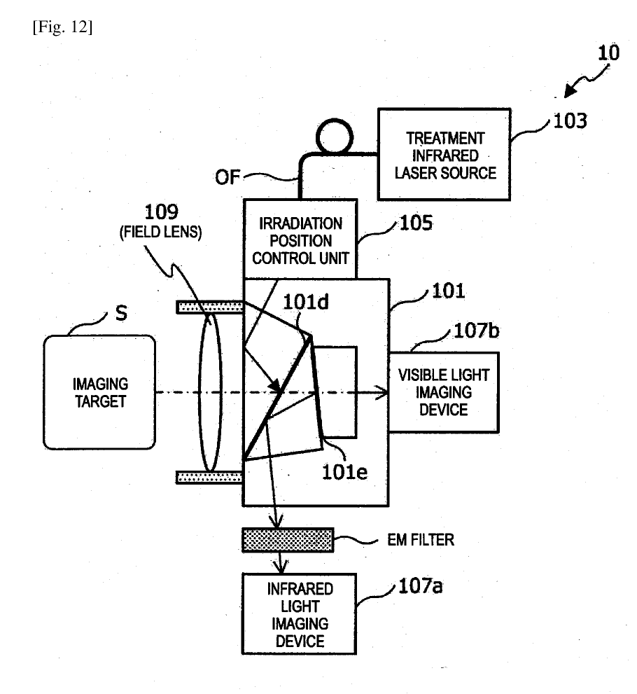

The optical system in the related configuration example is schematically illustrated in FIG. 12. In the imaging apparatus 10 of the present configuration example, the irradiation position control unit 105 is provided at the port A of the branching optical system 101, an EM filter that absorbs near-infrared light applied to the dye and an infrared light imaging device are provided as the first imaging device 107a at the port B of the branching optical system 101, and a visible light imaging device is provided at the port C of the branching optical system 101. In addition, a polarizing beam splitter (PBS) is provided at the first joint surface 101d, and a filter that transmits visible light and reflects infrared light (e.g., a filter that transmits light having a wavelength of 700 nm or lower and reflects light having a wavelength of higher than 700 nm) is provided as the wavelength selective filter at the second joint surface 101e. Further, a treatment infrared laser source which emits a treatment infrared laser beam absorbed in the dye infiltrated into an affected area is provided as the irradiation light source unit 103. To radiate the treatment infrared laser more uniformly, a field lens is provided as the optical device 109 between the branching optical system 101 and the imaging target S.

In the imaging apparatus illustrated in FIG. 12, visible light from the imaging target passes through the first joint surface 101d and the second joint surface 101e and then is imaged by the visible light imaging device. Infrared light from the imaging target passes through the first joint surface 101d and then is reflected at the second joint surface 101e. Thereafter, the reflected infrared light is imaged by the infrared light imaging device after irradiation light from the treatment infrared laser source has been removed by the EM filter.

In addition, polarization of the infrared laser emitted from the treatment infrared laser source is controlled such that the infrared laser beam is reflected by the polarizing beam splitter at the first joint surface 101d, and the irradiation position of the infrared laser is controlled by the irradiation position control unit 105 such that the infrared laser beam is applied to a desired region. The infrared laser beam of which the irradiation position has been controlled is reflected at the first joint surface 101d and applied to the affected area for which PIT is performed.

Examples of the configuration of the imaging apparatus 10 according to the present embodiment have been described in detail with reference to FIGS. 6 to 12.

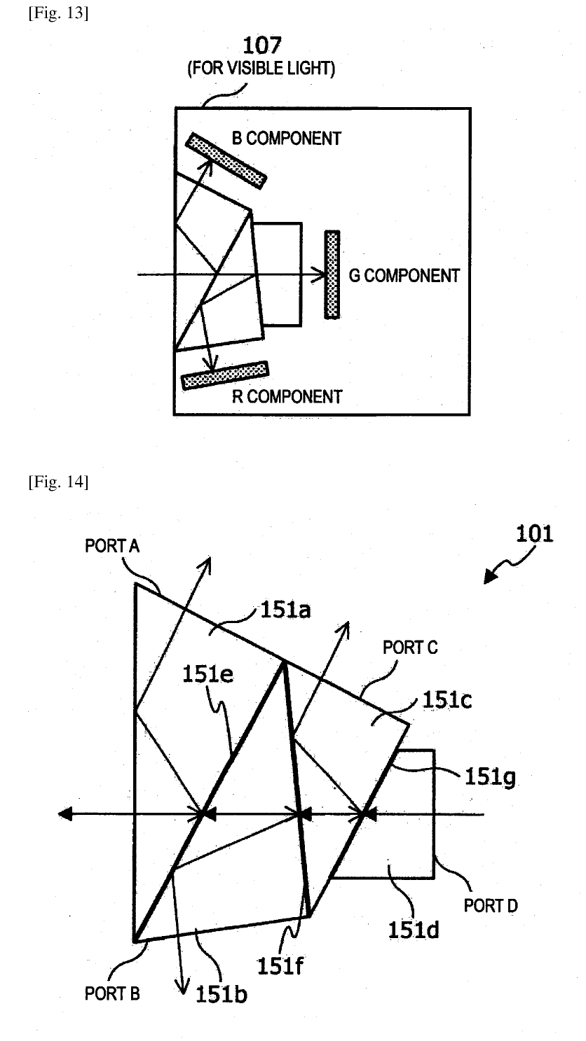

In the examples of the configuration of the imaging apparatus 10 according to the present embodiment, described with reference to FIGS. 6 to 12, while any imaging device may be used as the visible light imaging device, for example, a visible light imaging device using a 3-plate spectral prism, as illustrated in FIG. 13, may also be used. When the 3-plate spectral prism as shown in FIG. 13 is used, visible light input to the prism can be split into an R component, a G component and a B component with high accuracy and thus a high-quality visible light observation image can be obtained.

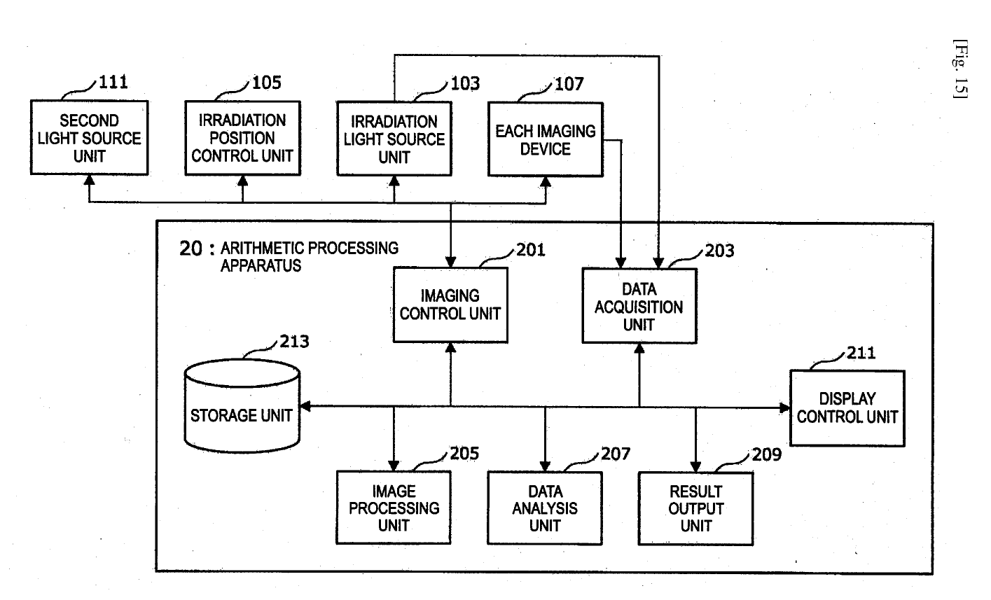

While the branching optical system which branches one optical path into three optical paths has been exemplified in the above description, it may be possible to branch one optical path into four optical paths using a branching optical system as illustrated in FIG. 14, for example. This branching optical system 101 includes a first optical prism 151a, a second optical prism 151b, a third optical prism 151c and a fourth optical prism 151d. In addition, the branching optical system 101 may realize four types of optical paths of ports A to D when functions of a first joint surface 151e, a second joint surface 151f and a third joint surface 151g are appropriately selected.

When one optical path is branched into five or more optical paths, it is possible to realize a desired number of branched optical paths by combining five or more optical prisms as in FIG. 14.

<Configuration of Arithmetic Processing Apparatus>

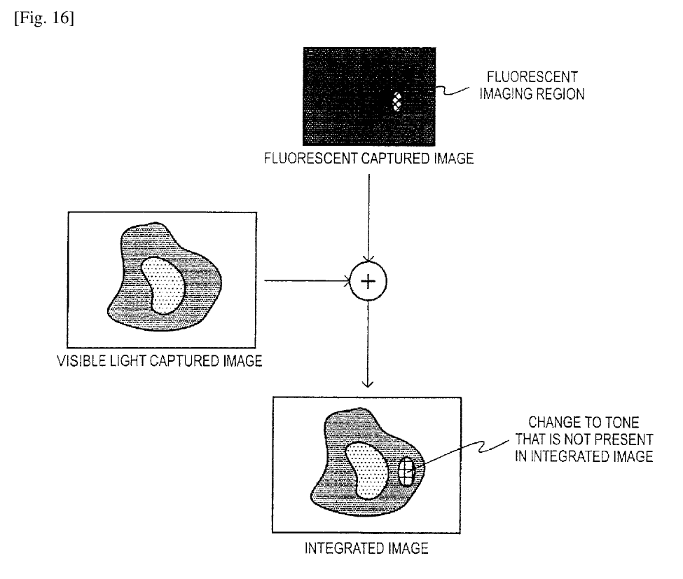

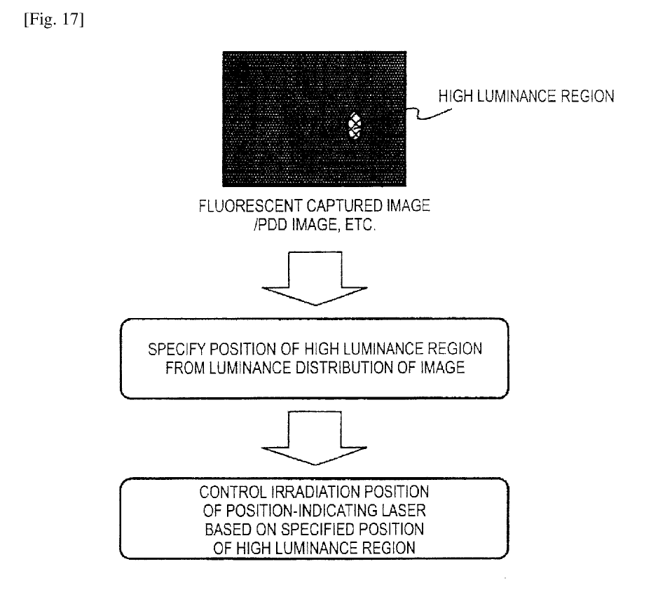

A configuration of the arithmetic processing apparatus 20 according to the present embodiment will be briefly described with reference to FIGS. 15 to 17. FIG. 15 is a block diagram schematically illustrating an example of the configuration of the arithmetic processing apparatus included in the imaging apparatus according to the present embodiment. FIG. 16 is an explanatory diagram schematically illustrating an example of image processing in the arithmetic processing apparatus according to the present embodiment and FIG. 17 is an explanatory diagram schematically illustrating an example of data analysis processing in the arithmetic processing apparatus according to the present embodiment.

As described above, the arithmetic processing apparatus 20 according to the present embodiment collectively controls the irradiation light source unit 103, the irradiation position control unit 105 and the at least one imaging device 107 and acquires image data of a captured image generated by the at least one imaging device 107. When the imaging apparatus 10 according to the present embodiment further includes the second light source unit 111, the arithmetic processing apparatus 20 may further control the second light source unit 111.

For example, the arithmetic processing apparatus 20 mainly includes an imaging control unit 201, a data acquisition unit 203, an image processing unit 205, a data analysis unit 207, a result output unit 209, a display control unit 211 and a storage unit 213, as shown in FIG. 15.

The imaging control unit 201 is realized by a central processing unit (CPU), a read only memory (ROM), a random access memory (RAM), a communication device and the like, for example. The imaging control unit 201 controls the irradiation light source unit 103, the irradiation position control unit 105 and the at least one imaging device 107 to be in desired states by respectively outputting predetermined control signals to the irradiation light source unit 103, the irradiation position control unit 105 and the at least one imaging device 107. When the imaging apparatus 10 according to the present embodiment further includes the second light source unit 111, the imaging control unit 201 may control an irradiation state of second light from the second light source unit 111 by outputting a predetermined control signal to the second light source unit 111.

The imaging control unit 201 may also be able to control the irradiation light source unit 103, the irradiation position control unit 105, the imaging device 107 and the like included in the imaging apparatus 10 in response to a user operation applied to the arithmetic processing apparatus 20 according to various methods. Accordingly, it may be possible to control the irradiation position of the position-indicating laser beam to be a desired position (e.g., a fluorescence emission region or the like) in configuration examples using the position-indicating laser source, for example, as described above.

In addition, the imaging control unit 201 may also be able to control the irradiation light source unit 103, the irradiation position control unit 105, the imaging device 107 and the like included in the imaging apparatus 10 on the basis of a data analysis result from the data analysis unit 207, which will be described below.

The data acquisition unit 203 is realized, for example, by a CPU, a ROM, a RAM, a communication device and the like. The data acquisition unit 203 acquires data output from the irradiation light source unit 103 (e.g., data of an optical tomographic image output from an OCT unit when the irradiation light source unit 103 is the OCT unit, or the like), data of various observation images output from each imaging device 107 and so on.

The image data acquired by the data acquisition unit 203 is output to the image processing unit 205 and the data analysis unit 207, which will be described below, as necessary and undergoes predetermined processing. Further, the image data acquired by the data acquisition unit 203 may be output to a user in various forms by the result output unit 209 which will be described below. Moreover, the data acquisition unit 203 may correlate acquired various image data with data such as dates and times when the image data is acquired and store the correlated data as history information in the storage unit 213 or the like.

The image processing unit 205 is realized by a CPU, a ROM, a RAM and the like, for example. The image processing unit 205 performs a predetermined image process on image data of a captured image (observation image) generated by the at least one imaging device 107. The image process performed by the image processing unit 205 is not particularly limited, and various known image processes may be performed.

When a plurality of imaging devices 107 are provided in the imaging apparatus 10 according to the present embodiment, the image processing unit 205 may generate an integrated image by integrating captured images generated by the respective imaging devices 107. For example, when a fluorescence imaging device and a visible light imaging device are provided in the imaging apparatus 10 according to the present embodiment, as illustrated in FIG. 16, the image processing unit 205 may generate an integrated image by integrating a fluorescence captured image generated by the fluorescence imaging device and a visible light captured image generated by the visible light imaging device.

When various captured images (e.g., a fluorescent captured image) are integrated with a visible light captured image, for example, it is desirable that the image processing unit 205 change the tone of a fluorescent imaged region in the fluorescent captured image to a tone that is not present in the integrated image. Accordingly, it is possible to prevent a user such as a doctor who refers to the integrated image from failing to notice the presence of the fluorescent imaged region because presence of the fluorescent imaged region is buried in the integrated image.

Further, the image processing unit 205 may acquire at least one of diagnosis images such as a mammography image, a CT image, an MRI image and an ultrasonic image of a patient corresponding to an imaging target from an external image server or the like and then generate integrated images by integrating the diagnosis image with various captured images generated by the imaging apparatus 10 according to the present embodiment.

After performing the aforementioned various image processes, the image processing unit 205 may output various processed images to the data analysis unit 207, the result output unit 209 and the like.

The data analysis unit 207 is realized by a CPU, a ROM, a RAM and the like, for example. The data analysis unit 207 performs various data analysis processes on image data of captured images generated by the at least one imaging device 107.

Data analysis processes performed by the data analysis unit 207 are not particularly limited and various known data analysis processes may be performed.

As one of such data analysis processes, for example, a process of calculating a distance to an imaging target on the basis of a time taken from when light is emitted from a TOF measurement light source to when light is detected by a TOF measurement imaging device when the imaging apparatus 10 according to the present embodiment has the TOF measurement function may be exemplified.

Further, when the imaging apparatus 10 according to the present embodiment includes the position-indicating laser source, the data analysis unit 207 may analyze a captured image (e.g., a fluorescent captured image, a PDD image or the like) generated by the at least one imaging device 107 to specify a portion (high luminance region) having a luminance value higher than a predetermined threshold value in the captured image, for example, as illustrated in FIG. 17.

When the position of the high luminance region is specified by analyzing the captured image, the data analysis unit 207 outputs an obtained specific result to the imaging control unit 201. The imaging control unit 201 may control the irradiation light source unit 103 and the irradiation position control unit 105 on the basis of the analysis result of the data analysis unit 207 to cause a laser beam from the position-indicating laser source to be emitted to the imaging target corresponding to the high luminance region. Accordingly, the position-indicating laser beam can be automatically applied to an appropriate position on the basis of the obtained captured image. For example, methods of specifying the high luminance region include a method of applying a laser beam to the outline of the high luminance region, a method of irradiating the whole high luminance region with a laser beam and so on.

The result output unit 209 is realized by a CPU, a ROM, a RAM, an output device, a communication device, etc., for example. The result output unit 209 outputs various captured images obtained by the imaging apparatus 10 according to the present embodiment, results of various image processes performed by the image processing unit 205, results of various data analysis processes performed by the data analysis unit 207 and the like to a user. For example, the result output unit 209 may output information about such results to the display control unit 211. Accordingly, the information about such results is output to a display unit (not shown) included in the arithmetic processing apparatus 20 and a display unit (e.g., an external monitor or the like) provided outside of the arithmetic processing apparatus 20. Further, the result output unit 209 may output information about obtained results as a printout or output the information to an external information processing apparatus, a server or the like as data.

The display control unit 211 is realized by a CPU, a ROM, a RAM, an output device and the like, for example. The display control unit 211 controls display when various results output from the result output unit 209 are displayed through an output device such as a display included in the arithmetic processing apparatus 20, an output device provided outside of the arithmetic processing apparatus 20 or the like. Accordingly, the user of the imaging apparatus 10 can recognize various results on the spot.

The storage unit 213 is an example of a storage device included in the arithmetic processing apparatus 20. The storage unit 213 appropriately stores various parameters that is necessary to be stored when the arithmetic processing apparatus 20 according to the present embodiment performs certain processing, status during processing and the like, or various databases, programs and the like. The storage unit 213 allows the imaging control unit 201, the data acquisition unit 203, the image processing unit 205, the data analysis unit 207, the result output unit 209, the display control unit 211 and the like to freely perform read/write processing.

An example of functions of the arithmetic processing apparatus 20 according to the present embodiment has been described. Each of the aforementioned components may be configured using a general-use member or circuit or using hardware specialized for the function thereof. Further, all functions of the components may be executed by a CPU or the like. Accordingly, a used configuration may be appropriately changed in response to a technology level when the present embodiment is performed.

Moreover, it is possible to produce a computer program for realizing the functions of the arithmetic processing apparatus according to the aforementioned present embodiment and install the computer program on a personal computer or the like. In addition, it is possible to provide a computer readable recording medium in which such a computer program is stored. The recording medium is a magnetic disk, an optical disc, a magneto-optical disc, a flash memory or the like, for example. The aforementioned computer program may be transmitted via a network, for example, without using the recording medium.

<<Example of Data Analysis Process by Data Analysis Unit 207>>

Hereinafter, an example of a data analysis process of the data analysis unit 207 in the arithmetic processing apparatus 20 will be described in detail by adopting a case in which a position-indicating laser source that emits visible light having a predetermined polarized component is installed as the irradiation light source unit 103 in the imaging apparatus 10 according to the present embodiment.

Conventionally, operation guide systems using projectors for projecting images and the like have been proposed. When images are projected using a projector in such an operation guide system, a shadowless lamp installed in an operating room has to be turned off. The reason for this will be briefly described blow.

When images are projected using a laser projector having picture quality of high vision (pixel number: 1920×1080), for example, images are projected by appropriately synchronizing an operation of scanning a laser beam emitted from a laser source of one point through two scan mirrors in the vertical direction and the horizontal direction with an on/off operation of the laser source. In addition, scanning of the laser beam is performed in the entire projectable range irrespective of contents of an image. In this case, when a point to which projection is performed corresponds to one pixel, for example, a time for which the laser beam is emitted becomes 1/(1920×1080) compared to a case in which all pixels are brightly displayed. That is, the luminance becomes 1/(1920×1080)=5.0×10⁻⁷ compared to a case in which the laser beam is indicated at one point without using a beam scanner.

Meanwhile, when an area (or outline) or the like is irradiated with a laser beam using the imaging apparatus 10 according to the present embodiment described above, it may be possible to draw images more brightly, compared to a case in which the general laser projector described above is used. Considering a case in which only one point (one pixel) is illuminated, for example, luminance of 5.0×10⁷ times that in a case in which the general laser projector is used may be obtained. Even when 100 points (100 pixels) are illuminated, luminance of 5.0×10⁵ times that in a case in which the general laser projector is used may be obtained. Accordingly, it can be said that luminance of a laser beam emitted from a laser source can be used more effectively as the number of illuminated points decreases. For this reason, it is possible to perform clear illumination even under a shadowless lamp by using the imaging apparatus 10 according to the present embodiment.

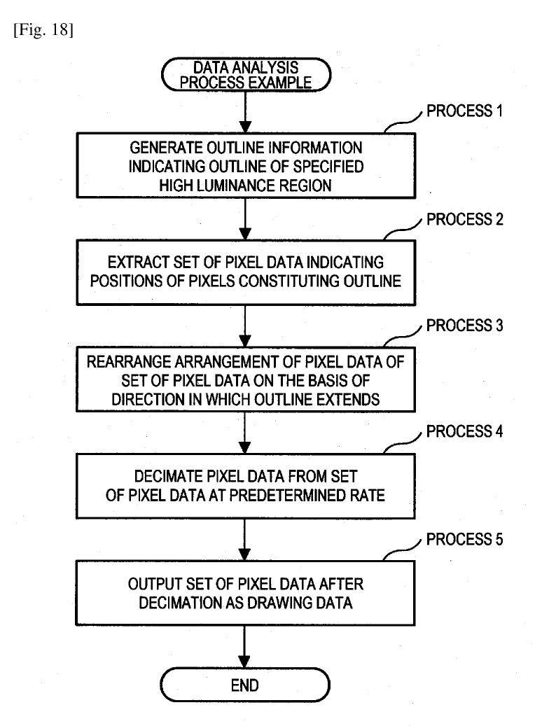

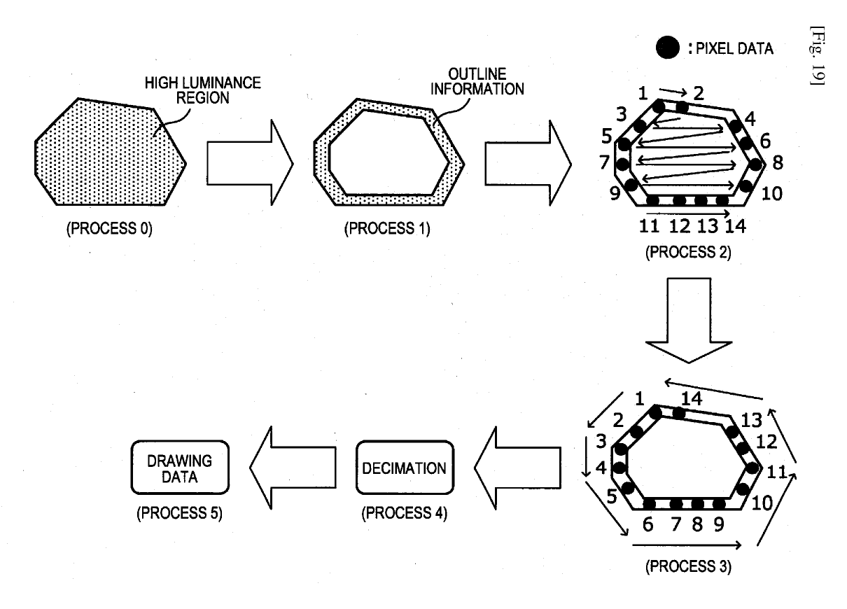

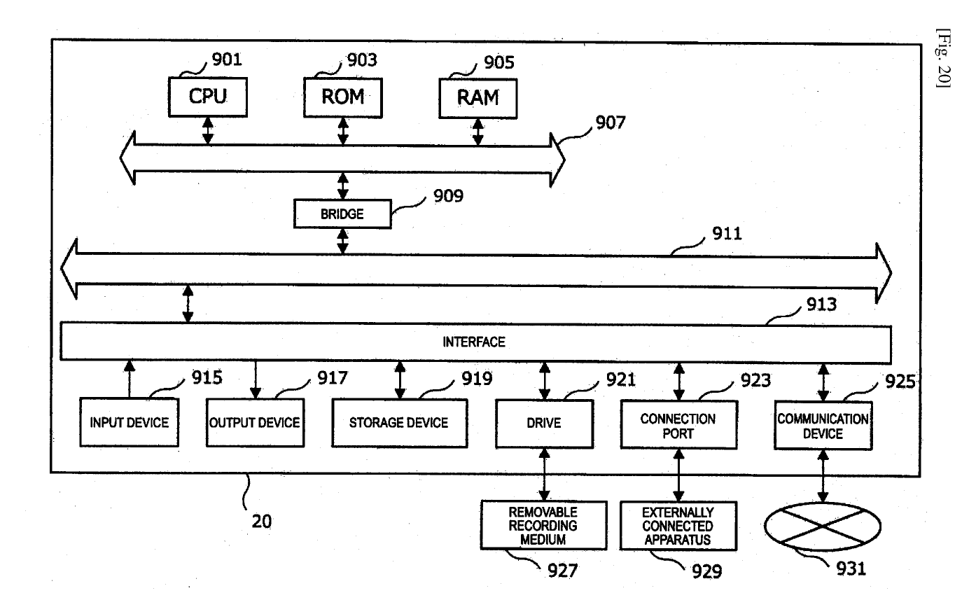

Hereinafter, an example of a data analysis process performed in the data analysis unit 207 will be described with reference to FIGS. 18 and 19 focusing on the circumstance in which the data analysis unit 207 analyzes a captured image generated by at least one imaging device to specify a portion having a luminance value higher than or equal to a predetermined threshold value in the captured image, and the imaging control unit 201 controls the irradiation light source unit 103 and the irradiation position control unit 105 to emit a laser beam from the position indication laser source to an imaging target corresponding to the portion having the luminance value higher than or equal to the predetermined threshold value. FIGS. 18 and 19 are explanatory diagrams schematically illustrating an example of a data analysis process in the arithmetic processing apparatus according to the present embodiment.

Prior to the data analysis process illustrated in FIG. 18, the data analysis unit 207 analyzes a captured image (e.g., a fluorescent captured image, a PDD image or the like) generated by at least one imaging device 107 to specify a portion (high luminance region) having a luminance value higher than or equal to a predetermined threshold value in such captured image, such as a position corresponding to a sentinel lymph node or the like (process 0). If the high luminance region can be specified in a fluorescent captured image, for example, using a laser beam having a wavelength of 808 nm, for example, the high luminance region corresponding to a sentinel lymph node can be easily specified by comparing the fluorescent captured image before the laser beam with the wavelength of 808 nm is radiated with the fluorescent captured image after the laser beam with the wavelength of 808 nm is radiated.

First of all, the data analysis unit 207 generates outline information indicating the outline of the specified high luminance region using an image related to the previously specified high luminance region (process 1). Although details of the process of generating such outline information is not particularly limited, the outline information corresponding to the outline form can be generated by binarizing the captured image including the high luminance region on the basis of a predetermined threshold value, and then uniformly magnifying the captured image at any magnification rate and comparing the captured images before and after magnification, for example.

Then, the data analysis unit 207 extracts a set of pixel data indicating positions of pixels constituting the outline using the generated outline information (process 2). An example illustrated in FIG. 19 shows a case in which data of 14 pixels is extracted from the outline information indicating the outline of the high luminance region. The set of pixel data extracted in this manner is arranged in a predetermined data arrangement (e.g., in ascending order or descending order based on coordinate values or the like) on the basis of coordinates indicating the pixel positions, for example. FIG. 19 schematically illustrates a case in which pixel data is arranged per row as an example of data arrangement, and numbers in the figure denote arrangement order of the pixel data for convenience.

Subsequently, the data analysis unit 207 rearranges the arrangement of the pixel data constituting the extracted set of pixel data on the basis of a direction in which the outline extends (process 3). By performing such rearranging of pixel data, the positions of the pixels constituting the outline are rearranged such that single-stroke writing is possible in the arrangement of the pixel data after rearrangement. FIG. 19 illustrates a case in which data of 14 pixels is sequentially rearranged counter-clockwise. According to such arrangement, a user of the imaging apparatus 10 can more easily recognize the outline of the high luminance region when the contour line is drawn.

After the rearrangement process as described above, the data analysis unit 207 decimates the pixel data from the rearranged set of pixel data at a predetermined rate (process 4). Accordingly, it is not necessary that more pixels than needed are irradiated with the laser beam from the position-indicating laser source when the contour line of the high luminance region is drawn, and thus the clear outline of the high luminance region can be drawn more accurately. Although the rate at which the pixel data is decimated is not particularly limited and is appropriately determined such that the luminance value of the laser beam does not decrease during drawing on the basis of the output of the used laser source, a normal size of the target high luminance region and the like, decimation of pixel data can be performed such that the number of data decreases to approximately ⅕.

Thereafter, the data analysis unit 207 outputs the set of decimated pixel data to the imaging control unit 201 as drawing data for drawing the high luminance region (process 5).

The imaging control unit 201 that has acquired the drawing data can draw the contour line of the high luminance region more clearly and in a state in which the user of the imaging apparatus 10 can easily recognize the outline by controlling the irradiation light source unit 103 and the irradiation position control unit 105 on the basis of the acquired drawing data.

An example of the data analysis process of the data analysis unit 207 in the arithmetic processing apparatus 20 according to the present embodiment has been described above in detail with reference to FIGS. 18 and 19.

The imaging apparatus 10 according to the present embodiment has been described in detail.

It is possible to realize utilization of a high-efficiency laser beam and to combine lights while saving a space by separating camera observation in the visible light band from a laser source or a laser measurement and analysis unit using the branching optical system through the aforementioned imaging apparatus 10 according to the present embodiment. In addition, it is possible to realize a small and lightweight camera scan unit for applications in which the resolution of a spot irradiated according to scanning is permitted to be lower than the resolution corresponding to a pixel in camera observation by inputting a beam narrower than the pupil diameter of an imaging optical system to the branching optical system through scanning.

Furthermore, it is possible to realize various medical observation apparatuses including a related imaging apparatus using the aforementioned imaging apparatus 10 according to the present embodiment. Such medical observation apparatuses are not particularly limited, and various medical observation apparatuses such as a microscope, an endoscope and an arthroscope may be exemplified. In addition, it is also possible to introduce the camera observation function in the visible light band in various diagnosis apparatuses and treatment apparatuses such as photodynamic diagnosis equipment, photodynamic treatment equipment and photo-immunotherapy equipment.

<Imaging Method>

An imaging method for using at least parts of at least three types of optical paths as an optical path for guiding light to an imaging target and an optical path for guiding light from the imaging target using the branching optical system 101 which coaxially branches incident light into at least three different types of optical paths, applying light having a predetermined wavelength, which has a controlled irradiation position, to the imaging target through a first optical path in the branching optical system 101, and guiding light from the imaging target to at least one imaging device through an optical path other than the first optical path in the branching optical system 101 is realized using the aforementioned imaging apparatus 10 according to the present embodiment.

<Hardware Configuration>

Next, the hardware configuration of the arithmetic processing apparatus 20 according to the embodiment of the present disclosure will be described in detail with reference to FIG. 18. FIG. 18 is a block diagram for illustrating the hardware configuration of the arithmetic processing apparatus 20 according to the embodiment of the present disclosure.

The arithmetic processing apparatus 20 mainly includes a CPU 901, a ROM 903, and a RAM 905. Furthermore, the arithmetic processing apparatus 20 also includes a host bus 907, a bridge 909, an external bus 911, an interface 913, an input device 915, an output device 917, a storage device 919, a drive 921, a connection port 923, and a communication device 925.

The CPU 901 serves as a main processing apparatus and a control device, and controls the overall operation or a part of the operation of the arithmetic processing apparatus 20 according to various programs recorded in the ROM 903, the RAM 905, the storage device 919, or a removable recording medium 927. The ROM 903 stores programs, operation parameters, and the like used by the CPU 901. The RAM 905 primarily stores programs used in execution of the CPU 901 and parameters and the like varying as appropriate during the execution. These are connected with each other via the host bus 907 configured from an internal bus such as a CPU bus or the like.

The host bus 907 is connected to the external bus 911 such as a PCI (Peripheral Component Interconnect/Interface) bus via the bridge 909.

The input device 915 is an operation means operated by a user, such as a mouse, a keyboard, a touch panel, buttons, a switch and a lever. Also, the input device 915 may be a remote control means (a so-called remote control) using, for example, infrared light or other radio waves, or may be an externally connected apparatus 929 such as a mobile phone or a PDA conforming to the operation of the arithmetic processing apparatus 20. Furthermore, the input device 915 generates an input signal based on, for example, information which is input by a user with the above operation means, and is configured from an input control circuit for outputting the input signal to the CPU 901. The user can input various data to the arithmetic processing apparatus 20 and can instruct the arithmetic processing apparatus 20 to perform processing by operating this input apparatus 915.

The output device 917 is configured from a device capable of visually or audibly notifying acquired information to a user. Examples of such device include display devices such as a CRT display device, a liquid crystal display device, a plasma display device, an EL display device and lamps, audio output devices such as a speaker and a headphone, a printer, a mobile phone, a facsimile machine, and the like. For example, the output device 917 outputs a result obtained by various processings performed by the arithmetic processing apparatus 20. More specifically, the display device displays, in the form of texts or images, a result obtained by various processes performed by the arithmetic processing apparatus 20. On the other hand, the audio output device converts an audio signal such as reproduced audio data and sound data into an analog signal, and outputs the analog signal.

The storage device 919 is a device for storing data configured as an example of a storage unit of the arithmetic processing apparatus 20 and is used to store data. The storage device 919 is configured from, for example, a magnetic storage device such as a HDD (Hard Disk Drive), a semiconductor storage device, an optical storage device, or a magneto-optical storage device. This storage device 919 stores programs to be executed by the CPU 901, various data, and various data obtained from the outside.

The drive 921 is a reader/writer for recording medium, and is embedded in the arithmetic processing apparatus 20 or attached externally thereto. The drive 921 reads information recorded in the attached removable recording medium 927 such as a magnetic disk, an optical disk, a magneto-optical disk, or a semiconductor memory, and outputs the read information to the RAM 905. Furthermore, the drive 921 can write in the attached removable recording medium 927 such as a magnetic disk, an optical disk, a magneto-optical disk, or a semiconductor memory. The removable recording medium 927 is, for example, a DVD medium, an HD-DVD medium, or a Blu-ray (registered trademark) medium. The removable recording medium 927 may be a CompactFlash (CF; registered trademark), a flash memory, an SD memory card (Secure Digital Memory Card), or the like. Alternatively, the removable recording medium 927 may be, for example, an IC card (Integrated Circuit Card) equipped with a non-contact IC chip or an electronic appliance.

The connection port 923 is a port for allowing devices to directly connect to the arithmetic processing apparatus 20. Examples of the connection port 923 include a USB (Universal Serial Bus) port, an IEEE1394 port, a SCSI (Small Computer System Interface) port, and the like. Other examples of the connection port 923 include an RS-232C port, an optical audio terminal, an HDMI (High-Definition Multimedia Interface) port, and the like. By the externally connected apparatus 929 connecting to this connection port 923, the arithmetic processing apparatus 20 directly obtains various data from the externally connected apparatus 929 and provides various data to the externally connected apparatus 929.

The communication device 925 is a communication interface configured from, for example, a communication device for connecting to a communication network 931. The communication device 925 is, for example, a wired or wireless LAN (Local Area Network), Bluetooth (registered trademark), a communication card for WUSB (Wireless USB), or the like. Alternatively, the communication device 925 may be a router for optical communication, a router for ADSL (Asymmetric Digital Subscriber Line), a modem for various communications, or the like. This communication device 925 can transmit and receive signals and the like in accordance with a predetermined protocol such as TCP/IP on the Internet and with other communication devices, for example. The communication network 931 connected to the communication device 925 is configured from a network and the like, which is connected via wire or wirelessly, and may be, for example, the Internet, a home LAN, infrared communication, radio wave communication, satellite communication, or the like.

Heretofore, an example of the hardware configuration capable of realizing the functions of the arithmetic processing apparatus 20 according to the embodiment of the present disclosure has been shown. Each of the structural elements described above may be configured using a general-purpose material, or may be configured from hardware dedicated to the function of each structural element. Accordingly, the hardware configuration to be used can be changed as appropriate according to the technical level at the time of carrying out the present embodiment.

It should be understood by those skilled in the art that various modifications, combinations, sub-combinations and alterations may occur depending on design requirements and other factors insofar as they are within the scope of the appended claims or the equivalents thereof.

Further, the effects described in this specification are merely illustrative or exemplified effects, and are not limitative. That is, with or in the place of the above effects, the technology according to the present disclosure may achieve other effects that are clear to those skilled in the art based on the description of this specification.

(1)

A medical imaging system, including:

an optical branching device having a plurality of optical paths for guiding light for imaging a target comprising a biotissue, each of the optical paths corresponding to an optical port connectable to an external device for imaging,

wherein at least one path of the plurality of optical paths is configured both to guide the irradiation light to the biotissue and to guide light from the biotissue, and

wherein the optical branching device includes a plurality of prisms and at least one joint surface.

(2)

The medical imaging system according to (1), wherein the at least one joint surface is at least one of a beam splitter (BS), a polarizing beam splitter (PCS) and a wavelength selective filter.

(3)

The medical imaging system according to (1)-(2), further including:

an imaging device connected to a port of the plurality of optical ports of the optical branching device.

(4)

The medical imaging system according to (1)-(3), further including:

a second imaging device connected to a second port of the plurality of optical ports of the optical branching device.

(5)

The medical imaging system according to (1)-(4), further including:

irradiation position control circuitry connected to a port of the plurality of optical ports of the optical branching device and configured to control a position of irradiation light emitted from an irradiation light source.

(6)

The medical imaging system according to (1)-(5), wherein the optical branching device is a bio-tissue excitation device.

(7)

The medical imaging system according to (1)-(6) wherein the optical branching device has three optical paths for guiding light

(8)

The medical imaging system according to (1)-(7), wherein the plurality of optical paths are at least partially coaxial.

(9)

The medical imaging system according to (1)-(8), further including:

a laser light source connected to a port of the plurality of optical ports of the optical branching device and configured to excite a specific area on the target.

(10)

The medical imaging system according to (1)-(9), further including:

an imaging device connected to a second port of the plurality of optical ports of the optical branching device and configured to image the excitation of the specific area on the target.

(11)

The medical imaging system according to (1)-(10), where in the imaging device is a fluorescence imaging device.

(12)

The medical imaging system according to (1)-(11), further including:

a field lens positioned between the optical branching device and the target.

(13)

The medical imaging system according to (1)-(12), further including:

processing circuitry configured to control a first imaging device connected to a first port of the plurality of optical ports of the optical branching device, a second imaging device connected to a second port of the plurality of optical ports of the optical branching device and irradiation position control circuitry connected to a third port of the plurality of optical ports of the optical branching device and configured to control a position of irradiation light emitted from an irradiation light source further controlled by the processing circuitry.

(14)

The medical imaging system according to (1)-(13), further including:

an excitation light source configured to excite the target;

a fluorescent imaging device connected to a first port of the plurality of optical ports of the optical branching device;

a visible imaging device connected to a second port of the plurality of optical ports of the optical branching device;

a laser source; and

irradiation position control circuitry connected to a third port of the plurality of optical ports of the optical branching device and configured to control a position of irradiation emitted from the laser source.

(15)

An optical branching device, including:

a plurality of optical paths for guiding light for imaging a target comprising a biotissue, each of the optical paths corresponding to an optical port connectable to an external device for imaging,

wherein at least one path of the plurality of optical paths is configured both to guide the irradiation light to the biotissue and to guide light from the biotissue, and

wherein the optical branching device includes a plurality of prisms and at least one joint surface.

(16)

The optical branching device according to (15), wherein the optical branching device has a plurality of faces, a face closest to the target being larger than any other of the plurality of faces.

(17)

The optical branching device according to (15)-(16), wherein the optical branching device has three optical paths for guiding light.

(18)

The optical branching device according to (15)-(17), wherein the plurality of optical paths are at least partially coaxial.

(19)

The medical imaging system according to (15)-(18), further including:

a plurality of light sources each having a different wavelength band including a visual wavelength laser source and a low coherence light source.

(20)

The medical imaging system according to (15)-(19), further including:

a time of flight (TOF) measurement imaging device connected to a port of the plurality of optical ports of the optical branching device.

(21)

The medical imaging system according to (15)-(20), further including:

an optical coherence tomography (OCT) device connected to a port of the plurality of optical ports of the optical branching device.

(1a)

An imaging apparatus including:

an irradiation light source unit configured to emit light having a predetermined wavelength to an imaging target;

an irradiation position control unit configured to control an irradiation position of irradiation light emitted from the irradiation light source unit on the imaging target;

at least one imaging device configured to image light from the imaging target; and

a branching optical system configured to coaxially branch incident light into at least three different types of optical paths,

wherein, in the branching optical system, at least parts of the at least three types of optical paths are used as an optical path for guiding the light to the imaging target and an optical path for guiding light from the imaging target, the irradiation light having the controlled irradiation position is emitted to the imaging target through a first optical path in the branching optical system, and the light from the imaging target is guided to the at least one imaging device through an optical path other than the first optical path in the branching optical system.

(2a)

The imaging apparatus according to (1a),

wherein the branching optical system is a spectral prism having at least three types of joined optical prisms, and

a joint surface between the optical prisms adjacent to each other serves as at least one of a beam splitter, a polarizing beam splitter and a wavelength selective filter to generate the at least three types of optical paths.

(3a)

The imaging apparatus according to (2a),

wherein the irradiation position control unit or the at least one imaging device is provided at an end of an optical path branched by the optical prisms.

(4a)

The imaging apparatus according to (3a),

wherein a position-indicating laser source configured to emit visible light having a predetermined polarized component is provided as the irradiation light source unit,

a fluorescence imaging device configured to image fluorescence from the imaging target and a visible light imaging device configured to image visible light are provided as the at least one imaging device,

a joint surface between the optical prism corresponding to an optical path at which the position-indicating laser source is provided and another optical prism neighboring the optical prism corresponding to the optical path at which the position-indicating laser source is provided serves as a polarizing beam splitter, and

a joint surface between the optical prism corresponding to an optical path at which the fluorescence imaging device is provided and the optical prism corresponding to an optical path at which the visible light imaging device is provided serves as a wavelength selective filter.

(5a)

The imaging apparatus according to (3a),

wherein an optical coherence tomography (OCT) unit configured to acquire an optical tomographic image of the imaging target by emitting irradiation light of an infrared wavelength band to the imaging target and detecting reflected light of the irradiation light of the infrared wavelength band from the imaging target is provided as the irradiation light source unit,

a visible light imaging device configured to image light belonging to a visible wavelength band is provided as the at least one imaging device, and

a joint surface between the optical prism corresponding to an optical path at which the OCT unit is provided and another optical prism neighboring the optical prism corresponding to the optical path at which the OCT unit is provided serves as a polarizing beam splitter.

(6a)

The imaging apparatus according to (3a),

wherein a quarter wave plate is provided between an optical prism closest to the imaging target and the imaging target,

a time-of-flight (TOF) measurement light source configured to emit irradiation light having a predetermined polarized component, used for a TOF method, is provided as the irradiation light source unit,

a TOF measurement imaging device and a visible light imaging device configured to image light belonging to a visible wavelength band are provided as the at least one imaging device,

a joint surface between the optical prism corresponding to an optical path at which the TOF measurement light source is provided and another optical prism neighboring the optical prism corresponding to the optical path at which the TOF measurement light source is provided and corresponding to an optical path at which the TOF measurement imaging device is provided serves as a polarizing beam splitter, and

a joint surface between the optical prism corresponding to the optical path at which the TOF measurement imaging device is provided and another optical prism corresponding to an optical path at which the visible light imaging device is provided serves as a wavelength selective filter.

(7a)

The imaging apparatus according to (3a),

wherein a position-indicating laser source configured to emit visible light having a predetermined polarized component is provided as the irradiation light source unit,

a first visible light imaging device configured to image fluorescence belonging to the visible wavelength band, generated from the imaging target when excitation light having a predetermined wavelength is emitted to the imaging target, and a second visible light imaging device configured to image visible light outside of the wavelength of the excitation light are provided as the at least one imaging device,

a joint surface between the optical prism corresponding to an optical path at which the position-indicating laser source is provided and another optical prism neighboring the optical prism corresponding to the optical path at which the position-indicating laser source is provided serves as a polarizing beam splitter, and

a joint surface between the optical prism corresponding to an optical path at which the first visible light imaging device is provided and the optical prism corresponding to an optical path at which the second visible light imaging device is provided serves as a beam splitter.

(8a)

The imaging apparatus according to (3a),

wherein a laser source configured to emit a laser beam having a predetermined polarized component and having a wavelength absorbed by the imaging target or a chemical material contained in the imaging target is provided as the irradiation light source unit,

a first visible light imaging device configured to image visible light and a second visible light imaging device configured to image visible light outside of the wavelength of the laser beam are provided as the at least one imaging device,

a joint surface between the optical prism corresponding to an optical path at which the laser source is provided and another optical prism neighboring the optical prism corresponding to the optical path at which the laser source is provided serves as a polarizing beam splitter, and

a joint surface between the optical prism corresponding to an optical path at which the first visible light imaging device is provided and the optical prism corresponding to an optical path at which the second visible light imaging device is provided serves as a beam splitter.

(9a)

The imaging apparatus according to (3a),

a laser source configured to emit a laser beam having a predetermined polarized component and belonging to an infrared wavelength band, absorbed by the imaging target or a chemical material contained in the imaging target, is provided as the irradiation light source unit,

an infrared light imaging device configured to image infrared light outside of the wavelength of the laser beam and a visible light imaging device configured to image visible light are provided as the at least one imaging device,

a joint surface between the optical prism corresponding to an optical path at which the laser source is provided and another optical prism neighboring the optical prism corresponding to the optical path at which the laser source is provided serves as a polarizing beam splitter, and

a joint surface between the optical prism corresponding to an optical path at which the infrared light imaging device is provided and the optical prism corresponding to an optical path at which the visible light imaging device is provided serves as a wavelength selective filter.

(10a)

The imaging apparatus according to any one of (1a) to (9a),

wherein the irradiation position control unit is a scanning unit having at least one of a galvanomirror and a MEMS mirror.

(11a)

The imaging apparatus according to any one of (1a) to (9a),

wherein the irradiation position control unit is a scanning unit configured to scan the irradiation light by controlling a position of an exit end of an optical fiber for guiding the irradiation light.

(12a)

The imaging apparatus according to any one of (1a) to (11a), further including

a second light source configured to emit second light different from the irradiation light,

wherein the second light is emitted to the imaging target without passing through the branching optical system.

(13a)

The imaging apparatus according to any one of (1a) to (12a), further including

an arithmetic processing apparatus configured to control the irradiation light source unit, the irradiation position control unit and the at least one imaging device and to acquire image data of captured images generated by the at least one imaging device, wherein the arithmetic processing apparatus includes an imaging control unit configured to control the irradiation light source unit, the irradiation position control unit and the at least one imaging device, and at least one of an image processing unit configured to perform a predetermined image process on the image data of the captured images generated by the at least one imaging device and a data analysis unit configured to perform a predetermined data analysis process on the image data of the captured images generated by the at least one imaging device.

(14a)

The imaging apparatus according to (13a),

wherein two or more of the imaging devices are provided as the at least one imaging device, and

the image processing unit generates an integrated image by integrating captured images generated by the respective imaging devices.

(15a)

The imaging apparatus according to (13a) or (14a),

wherein a position-indicating laser source configured to emit visible light having a predetermined polarized component is provided as the irradiation light source unit,

the data analysis unit analyzes the captured images generated by the at least one imaging device to specify portions having luminance values higher than a predetermined threshold value in the captured images, and

the imaging control unit controls the irradiation light source unit and the irradiation position control unit on the basis of an analysis result of the data analysis unit to cause a laser beam from the position-indicating laser source to be emitted to the imaging target corresponding to the portions having the luminance values higher than the predetermined threshold value.

(16a)

The imaging apparatus according to any one of (1a) to (15a),

wherein the branching optical system is optically connected to an endoscope or an arthroscope, and

the imaging target is imaged through the endoscope or the arthroscope.

(17a)

An imaging method including:

using at least parts of at least three types of optical paths different from each other as an optical path for guiding light to an imaging target and an optical path for guiding light from the imaging target, using a branching optical system that coaxially branches incident light into the at least three types of optical paths, and

applying light having a predetermined wavelength and having a controlled irradiation position to the imaging target through a first optical path in the branching optical system, and guiding light from the imaging target to at least one imaging device through an optical path other than the first optical path in the branching optical system.

(18a)

Medical observation equipment including at least an imaging apparatus, the imaging apparatus including:

an irradiation light source unit configured to emit light having a predetermined wavelength to biotissue;

an irradiation position control unit configured to control an irradiation position of irradiation light emitted from the irradiation light source unit on the biotissue;

at least one imaging device configured to image light from the biotissue; and

a branching optical system configured to coaxially branch incident light into at least three different types of optical paths,

wherein, in the branching optical system, at least parts of the at least three types of optical paths are used as an optical path for guiding the light to the biotissue and an optical path for guiding light from the biotissue, the irradiation light having the controlled irradiation position is emitted to the biotissue through a first optical path in the branching optical system, and the light from the biotissue is guided to the at least one imaging device through an optical path other than the first optical path in the branching optical system.

### REFERENCE SIGNS LIST

10 imaging apparatus
20 arithmetic processing apparatus
101 branching optical system
103 irradiation light source unit
105 irradiation position control unit
107 imaging device
109 optical device
111 second light source unit
201 imaging control unit
203 data acquisition unit
205 image processing unit
207 data analysis unit
209 result output unit
211 display control unit
213 storage unit

1. A medical imaging system, comprising:
an optical branching device having a plurality of optical paths for guiding light for imaging a target comprising a biotissue, each of the optical paths corresponding to an optical port connectable to an external device for imaging,
wherein at least one path of the plurality of optical paths is configured both to guide the irradiation light to the biotissue and to guide light from the biotissue, and
wherein the optical branching device includes a plurality of prisms and at least one joint surface.

2. The medical imaging system according to claim 1, wherein the at least one joint surface is at least one of a beam splitter (BS), a polarizing beam splitter (PCS) and a wavelength selective filter.

3. The medical imaging system according to claim 1, further comprising:
an imaging device connected to a port of the plurality of optical ports of the optical branching device.

4. The medical imaging system according to claim 3, further comprising:
a second imaging device connected to a second port of the plurality of optical ports of the optical branching device.

5. The medical imaging system according to claim 1, further comprising:
irradiation position control circuitry connected to a port of the plurality of optical ports of the optical branching device and configured to control a position of irradiation light emitted from an irradiation light source.

6. The medical imaging system according to claim 1, wherein the optical branching device is a bio-tissue excitation device.

7. The medical imaging system according to claim 1, wherein the optical branching device has three optical paths for guiding light

8. The medical imaging system according to claim 1, wherein the plurality of optical paths are at least partially coaxial.

9. The medical imaging system according to claim 1, further comprising:
a laser light source connected to a port of the plurality of optical ports of the optical branching device and configured to excite a specific area on the target.

10. The medical imaging system according to claim 9, further comprising:
an imaging device connected to a second port of the plurality of optical ports of the optical branching device and configured to image the excitation of the specific area on the target.

11. The medical imaging system according to claim 10, where in the imaging device is a fluorescence imaging device.

12. The medical imaging system according to claim 1, further comprising:
a field lens positioned between the optical branching device and the target.

13. The medical imaging system according to claim 1, further comprising:
processing circuitry configured to control a first imaging device connected to a first port of the plurality of optical ports of the optical branching device, a second imaging device connected to a second port of the plurality of optical ports of the optical branching device and irradiation position control circuitry connected to a third port of the plurality of optical ports of the optical branching device and configured to control a position of irradiation light emitted from an irradiation light source further controlled by the processing circuitry.

14. The medical imaging system according to claim 1, further comprising:
an excitation light source configured to excite the target;
a fluorescent imaging device connected to a first port of the plurality of optical ports of the optical branching device;
a visible imaging device connected to a second port of the plurality of optical ports of the optical branching device;
a laser source; and
irradiation position control circuitry connected to a third port of the plurality of optical ports of the optical branching device and configured to control a position of irradiation emitted from the laser source.

15. An optical branching device, comprising:
a plurality of optical paths for guiding light for imaging a target comprising a biotissue, each of the optical paths corresponding to an optical port connectable to an external device for imaging,
wherein at least one path of the plurality of optical paths is configured both to guide the irradiation light to the biotissue and to guide light from the biotissue, and
wherein the optical branching device includes a plurality of prisms and at least one joint surface.

16. The optical branching device according to claim 15, wherein the optical branching device has a plurality of faces, a face closest to the target being larger than any other of the plurality of faces.

17. The optical branching device according to claim 15, wherein the optical branching device has three optical paths for guiding light.

18. The optical branching device according to claim 15, wherein the plurality of optical paths are at least partially coaxial.

19. The medical imaging system according to claim 1, further comprising:
a plurality of light sources each having a different wavelength band including a visual wavelength laser source and a low coherence light source.

20. The medical imaging system according to claim 1, further comprising:
a time of flight (TOF) measurement imaging device connected to a port of the plurality of optical ports of the optical branching device.

21. The medical imaging system according to claim 1, further comprising:
an optical coherence tomography (OCT) device connected to a port of the plurality of optical ports of the optical branching device.
